(19) United States
(12) Patent Application Publication
(10) Pub. No.: US 2021/0393522 A1
(43) Pub. Date: Dec. 23, 2021

(54) COMPOSITIONS FOR SOLUBILIZING WATER-INSOLUBLE ACTIVE INGREDIENTS

(71) Applicant: Neha Chavan, West Hartford, CT (US)

(72) Inventor: Neha Chavan, West Hartford, CT (US)

(21) Appl. No.: 17/351,159

(22) Filed: Jun. 17, 2021

Related U.S. Application Data
(60) Provisional application No. 63/041,839, filed on Jun. 20, 2020.

(51) Int. Cl. A61K 9/10; A61K 47/14; A61K 45/06

(52) U.S. Cl. CPC A61K 9/10; A61K 45/06; A61K 47/14

(57) ABSTRACT

The present invention provides compositions for solubilizing water-insoluble and poorly water-soluble or poorly permeable active ingredients such as drugs, nutritional supplements, and essential oils. The compositions are useful for administration of the active ingredient to the subject via various routes and provide good bioavailability of the active. The compositions are generally clear and non-turbid and are useful, for example, for preparing formulations of cannabinoid compounds.

### CROSS-REFERENCE TO RELATED APPLICATIONS

This application claims priority under 35 U.S.C. § 119(e) to U.S. Provisional Application No. 63/041,839, filed Jun. 20, 2020, which is hereby incorporated by reference in its entirety herein.

### FIELD OF THE INVENTION

The present invention provides compositions for solubilizing water-insoluble and poorly water-soluble active ingredients such as drugs, botanical and animal extracts, nutritional supplements, and essential oils. The scope of these delivery systems also extends to increasing the absorption of the active ingredients through epithelial or mucosal membranes in the body of a subject. The compositions can be incorporated into a variety of (1) solid dosage forms such as capsules, tablets, powders, films, suppositories, etc.; (2) semi-solid dosage forms such as topical creams, salves, gels, transdermal patches, etc.; and (3) liquid dosage forms such as oral-mucosal liquids, beverages, beverage additives, intranasal liquids, spray mists for oral-mucosal and intranasal delivery, and injectable compositions. The liquid compositions are generally clear and non-turbid and are useful for formulating essential oils such as cannabinoid compounds.

### BACKGROUND OF THE INVENTION

In the pharmaceutical, nutraceutical, cosmetic, and food industries, there is a continuing need to develop compositions and methods for solubilizing water-insoluble active ingredients. The reasons for this need are that: (1) many compositions of consumer products and pharmaceuticals are water-based, (2) the human body is comprised mostly of water, i.e. about 60% by weight, and (3) the human bloodstream, which carries medicines and nutrients throughout the body, is over 90% by weight water. Therefore, the more completely a nutrient, compound, or medicine is solubilized in water, the more efficiently it can be absorbed into the human body. Solubility improvements also ultimately lead to improved bio-availability for most medicines, nutrients, molecules, compounds, and extracts that humans consume. The same considerations also apply to many compositions for administration to animals such as mammals and birds.

A variety of plant species have therapeutic benefits when consumed at appropriate dosages as foods, nutritional supplements, or as medicines. Many cultures, both modern and ancient, have vast libraries of traditional herbs recognized for their medicinal purposes. For example, cannabis is one of the oldest medicinal plants known to mankind, and the list of recognized medicinal botanicals and essential oils includes over 30,000 herbs.

Ayurveda and Siddha, are Indian systems of medicine, believed to be the oldest medical systems in the world and is based on a holistic approach to physical and mental health. Ayurvedic medicine remains one of India's most widely used systems for human wellbeing, and relies on products mainly derived from plant sources, although it also includes products derived from animal, metal, and mineral sources. Diet, exercise, and lifestyle are also key aspects of the Ayurvedic system of health and wellbeing. Cannabis in particular has been widely used in traditional Indian medicine for thousands of years and has been increasingly recognized in Western medicine for its utility in treating and healing a wide variety of health conditions such as cancer, anxiety, depression, pain, seizures, etc. See, Grotenhermen, F. (2005). Cannabinoids. Current Drug Targets-CNS & Neurological Disorders (4.5), 507-530; and Guzmán, M. (2003). Cannabinoids: potential anticancer agents. Nature reviews cancer (3.10), 745-755. Therefore, it would be highly desirable to deliver bioavailable compositions of cannabis extracts and other active ingredients.

By some estimates, over 70% of new pharmaceutical drug molecules are classified as poorly water-soluble and/or poorly permeable. See, Di, L. E. (2009). Drug-like property concepts in pharmaceutical design. Current pharmaceutical design, 15.19, 2184-2194. The high throughput systems, primarily used in the pharmaceutical industry to identify and develop compounds as therapeutic drugs focus on enhancing a molecule's ability to bind to a target receptor, but often ignore solubility considerations, resulting in active drugs that are poorly water-soluble and poorly permeable. However, water solubility and permeability are important components determining the pharmacokinetic and bioavailability of drugs.

Water solubility for oral and mucosal delivery is significant because the gastrointestinal tract comprises aqueous gastric liquids. The solubilization of actives in the gastrointestinal fluids ensures that the active ingredients are available in small particle size at the site of absorption, thereby leading to the efficient absorption of the active through the gut membrane and into the blood stream for transport throughout the body.

A potential formulation solution for delivery of water-insoluble or poorly water soluble actives is to microencapsulate the active for dispersion in an aqueous system. However, turbid microencapsulated compositions have extremely short shelf stability that can result in precipitation of the active ingredients from the compositions upon storage. Therefore, consumers do not receive the intended therapeutic dosage, which can be inconvenient or even harmful. Also, existing turbid microencapsulated systems in the cannabis industry generally are limited to specific isolated molecules, e.g., cannabidiol (CBD) or tetrahydrocannabinol (THC), rather than full-spectrum cannabis extracts containing all of the organic cannabinoids, terpenes, and flavonoids found naturally in the plant. Existing formulations claiming to use full-spectrum hemp are, in fact, filtering out much of the original plant matter in their processing. Therefore, the full benefits of a full-spectrum cannabis extract are not being provided. It would be highly desirable to provide improved formulations for delivering full-spectrum hemp and cannabis, which have been shown to provide superior clinical benefits when compared to isolated CBD or THC. See, Gallily, R. Z. (2015). Overcoming the bell-shaped dose-response of cannabidiol by using cannabis extract enriched in cannabidiol. Pharmacology & Pharmacy (6.02), 75.

Upon oral ingestion of oil or turbid compositions, the body releases lipases to catalyze the break-down of the lipid components. Then, bile salts, which are the natural solubilizer compounds found in the body, solubilize the poorly water-soluble actives. However, the extent to which bile salts can resolubilize actives is limited and concentration dependent, thereby limiting the absorption of the active ingredients through the gastrointestinal membrane. For example, studies have shown that as little as 4% of the total CBD in oil-based delivery systems is absorbed by humans. See, Agurell S, C. S. (1981). Interaction of THC with cannabinol and cannabidiol following oral administration in man. Assay of cannabinol and cannabidiol by mass fragmentography. Experimentia(37), 1090-1092.

Most currently marketed cannabinoid products for oral ingestion use CBD solubilized in an oil base or in a turbid microencapsulated composition. The disadvantages associated with both of these types of compositions are that the active ingredients are limited by:
1) poor solubilization,
2) poor permeation through mucosal or epithelial membranes,
3) inconsistent dosing,
4) inconsistent potency in each product,
5) inconsistent and delayed onset of action,
6) extensive first pass metabolism,
7) limited scope of product applications,
8) low aesthetics and consumer appeal due to cloudy and murky looking solutions,
9) inability to solubilize in water the complete organic, raw plant extracts, such as full-spectrum hemp or cannabis crude oil (and thereby limiting aqueous formulations to specific isolates such as CBD, THC, or to highly processed and filtered hemp extracts), and
10) low bioavailability, which poses the disadvantage of requiring higher doses which can increase the risk of side effects.

Furthermore, existing CBD products for topical, transdermal, or mucosal applications are generally using a CBD dispersed oil rather than full-spectrum water soluble CBD. The disadvantages associated with these compositions are that the CBD active ingredient has:
1) poor permeation through the skin barrier,
2) inconsistent dosing,
3) inconsistent potency in each product,
4) limited product applications,
5) inability to solubilize in water the complete organic, raw plant extracts, such as full-spectrum hemp or cannabis crude oil, and
6) low bioavailability, which poses the disadvantage of requiring higher doses which can increase the risk of side effects.

The present invention addresses these drawbacks associated with the oil-based compositions and turbid microencapsulated compositions currently on the market. An advantage of the present invention is that it seamlessly and simultaneously provides a delivery platform for:
1) water-insoluble and poorly water-soluble molecules, including cannabinoids,
2) water-insoluble and poorly water-soluble extracts of botanical or animal origin, including full-spectrum cannabis extracts,
3) poorly permeable molecules, and
4) any combination of the above.

The present invention solves the limitations of current so-called solubilization methods and compositions by creating compositions that are clear, highly shelf stable, and easily used in a wide array of product applications. The present invention therefore provides technology for the creation of water-soluble and highly permeable product compositions for various dosage forms including oral, topical, and transdermal applications. The present invention can provide for an effective platform to deliver therapeutic molecules and/or botanical extracts, such as cannabinoid compounds and full-spectrum cannabis extracts.

### SUMMARY OF THE INVENTION

The present invention provides compositions for solubilizing water-insoluble and poorly water-soluble active ingredients such as drugs, nutritional supplements, and essential oils.

In further embodiments, the scope of these delivery systems also extend to increasing the absorption of these compounds through epithelial or mucosal membranes in the body. Some compositions have ingredients known to bypass hepatic first pass metabolism as well. An object of the present invention is to incorporate water-insoluble and/or poorly water-soluble actives, and when also desired, water-soluble actives, in the same composition, thereby improving the water solubility and absorption of the active ingredients into the body. The compositions explained in the present invention can be administered via a wide variety of routes, including, for example, oral, mucosal, topical, or invasive routes. Invasive routes may include parenteral routes, such as epidural, intracerebral, intracerebroventricular, intra-arterial, intra-articular, intracardiac, intradermal, intralesional, intramuscular, intraocular, intraosseous, intraperitoneal, intrathecal, subcutaneous, and others known to a person of skill in the art. The compositions of the current invention can be incorporated into a variety of dosage forms such as capsules, tablets, oral powders optionally dissolved in water or an aqueous carrier, oral-mucosal liquids, beverages, beverage additives, intranasal liquids, spray mists for oral-mucosal and intranasal delivery, topical products, transdermal patches, suppositories, and injectable formulations. The compositions in the current invention can aid in reducing the dose as well as side effects of the actives in the existing formulations.

In further embodiments, the present invention provides compositions comprising active ingredients including cannabinoids and full-spectrum cannabis extracts.

In some embodiments, the present invention provides a preconcentrate composition for solubilizing, dispersing, or emulsifying a water-insoluble or poorly water-soluble active ingredient in an aqueous carrier, said preconcentrate composition, comprising:
a. a lipophilic component having an hydrophilic-lipophilic balance (HLB) value of zero to about 7, and
b. a surface-active agent having an HLB value from about 10 to about 13.

In a further embodiment, the lipophilic component comprises from about 0.1% to about 99.9%, or from about 1% to about 80%, or from about 5% to about 50%, or from about 10% to about 40%, or from about 10% to about 25% by weight of the pre-concentrate.

In a further embodiment, the surface active agent comprises from about 0.1% to about 99.9%, or from about 1% to about 80%, or from about 5% to about 50%, or from about 10% to about 40%, or from about 10% to about 25% by weight of the pre-concentrate.

In a further embodiment, said preconcentrate may be combined with said water-insoluble or poorly water-soluble active ingredient to form a concentrate having a HLB value from about 7 to about 10.

In a further embodiment said resultant concentrate is capable of providing a dispersion in said aqueous carrier, wherein the dispersion comprises particles formed from the concentrate.

In a further embodiment, said resultant concentrate is capable of providing a visibly clear aqueous composition when combined with an aqueous carrier.

In a further embodiment, the particles formed from the dispersion of the concentrate have a particle size distribution with a mode less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the particles formed from the dispersion of the concentrate have a particle size distribution with a mode in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the particles formed from the dispersion of the concentrate have a particle size distribution with a D50 value less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the particles formed from the dispersion of the concentrate have a particle size distribution with a D50 value in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the lipophilic component is selected from the group consisting of plant-based oils, glycerides, waxes, alcohols, hydroalcoholic mixtures, whole and fractionated oil forms of any of the foregoing, and mixtures thereof.

In a further embodiment, the lipophilic component is a plant-based oil, or a whole or fractionated oil form thereof, and mixtures thereof.

In a further embodiment, the plant-based oil is selected from the group consisting of almond oil, avocado oil, borage oil, brazil nut oil, cannabis oil, cannabis-seed oil, canola oil, cashew oil, castor oil, chia seed oil, cocoa butter oil, coconut oil, corn oil, cottonseed oil, flaxseed oil, grape seed oil, hemp seed oil, linseed oil, mustard oil, olive oil, palm oil, peanut oil, pecan oil, peppermint oil, perilla oil, poppy seed oil, rapeseed oil, rice bran oil, safflower oil, sesame oil, sesame seed oil, soybean oil, sunflower oil, vigna munga oil, walnut oil, whole and fractionated oil forms of any of the foregoing, and mixtures thereof

In a further embodiment, the lipophilic component is a glyceride and mixtures thereof.

In a further embodiment, the glyceride is selected from monoglycerides, diglycerides, triglycerides, and mixtures thereof.

In a further embodiment, the glyceride is selected from monoglycerides, diglycerides, and triglycerides of C6 to C30 carboxylic acids, and mixtures thereof, wherein the C6 to C30 carboxylic acids are selected from fully saturated carboxylic acids, carboxylic acids having 1, 2, or 3 unsaturated carbon-carbon bonds which can be variously positioned along the carbon skeleton of the carboxylic acid and which can each individually have a cis or trans isomeric configuration, and wherein the C6 to C30 carboxylic acids can be optionally substituted with one or more hydroxyl groups, amino groups, or carbonyl groups, and combinations of these groups.

In a further embodiment, the glycerides are selected from the group consisting of those corresponding to the following CAS registry numbers CAS 92045-31-3, CAS 91744-32-0, CAS 85536-07-8, CAS 91052-28-7, CAS 91744-09-1, CAS 91744-13-7, CAS 24529-88-2; CAS 85251-77-0, CAS 84244-35-9, CAS 85536-06-7, CAS 91744-20-6, CAS 122-32-7, CAS 25496-72-4, and mixtures thereof.

In a further embodiment, the glycerides are selected from the group consisting of cocoglycerides; glyceryl caprate (C8-10 mono, di, and triglycerides); glycerides, C14-18 and C16-18-unsaturated mono-, di- and tri-; glycerides C16-18 and C18-unsaturated mono-; glycerides, C14-18 and C16-22-unsaturated mono- and di-; glycerides C16-18 mono- and di-; (1-hexadecanoyloxy-3-hydroxypropan-2-yl) octadecenoate; glycerides, C8-18; glycerides, C16-18 and C18-unsaturated mono-, di and tri-; 1,2,3-tri(cis-9-octadecenoyl)glycerol; glyceryl monooleate; and combinations thereof.

In a further embodiment, the surface-active agent comprises a hydrophilic head group and one or more side chains selected from C10-C30 fatty acids, wherein the surface-active agent is ethoxylated, propoxylated, or mixed ethoxylated/propoxylated.

In a further embodiment, the one or more C10-C30 fatty acids each have at least one hydroxy substituent.

In a further embodiment, at least one of the hydroxy substituents of the C10-C30 fatty acids is ethoxylated, propoxylated, or mixed ethoxylated/propoxylated.

In a further embodiment, the one or more C10-C30 fatty acids each have at least one carbon-carbon unsaturated bond.

In a further embodiment, the one or more C10-C30 fatty acids have at least one carbon-carbon unsaturated bond in the cis configuration.

In a further embodiment, the hydrophilic head group is selected from the group consisting of aliphatic alcohols, aliphatic polyhydric alcohols, saccharides, disaccharides, aliphatic amines, aliphatic polyamines, aliphatic amino alcohols, aliphatic amino polyhydric alcohols, aliphatic polyamino alcohols, aliphatic polyamino polyhydric alcohols, and combinations thereof.

In a further embodiment, the hydrophilic head group is selected from the group consisting of ethylene glycol, propylene glycol, 1,3-propane diol, 1,2-butane diol, 1,3-butane diol, 1,4-butane diol, glycerol, glyceraldehyde, 1-hydroxy-2-amino ethane, and combinations thereof.

In a further embodiment, the head group is glycerol.

In a further embodiment, the surface active agent is selected from a monoglyceride, a diglyceride, a triglyceride, and combinations thereof.

In a further embodiment, the surface-active agent comprises a mono-, di-, or triglyceride of a C10-C30 fatty acid wherein each C10-C30 fatty acid is independently modified with one or more ethoxy groups, one or more propoxy groups, or a mixture of one or more ethoxy and propoxy groups.

In a further embodiment, the hydrophilic head group is covalently bonded to the one or more C10-C30 fatty acids directly via an ester linkage to the C10-C30 fatty acid, and wherein at least one of the hydroxy substituents of at least one of the C10-C30 fatty acid chains is independently modified with one or more ethoxy groups, propoxy groups, or a mixture of one or more ethoxy and propoxy groups.

In a further embodiment, the hydrophilic head group is covalently bonded to the one or more C10-C30 fatty acids indirectly via an ester or ether, linkage to an intervening ethoxy, propopxy, or mixed ethoxy/propoxy group.

In a further embodiment, the hydrophilic head group is covalently bonded to the one or more C10-C30 fatty acids indirectly via an ester or ether linkage to an intervening ethoxy, propopxy, or mixed ethoxy/propoxy group, and wherein at least one of the hydroxy substituents of at least one of the C10-C30 fatty acid chains is modified with one or more ethoxy groups, propoxy groups, or mixed ethoxy and propoxy groups.

In a further embodiment, the surface active agent is selected from the group consisting of CAS 61788-85-0, CAS 188734-82-9, CAS 57176-33-7, CAS 70142-34-6, CAS 68953-20-8, CAS 31835-02-6, CAS 13039-40-2, CAS 61791-12-6, CAS 854374-08-6, CAS 122636-35-5, CAS 122636-36-6, CAS 9005-64-5, CAS 9005-65-6, CAS 145-42-6, CAS 388610-12-6, and mixtures thereof.

In a further embodiment, the surface-active agent is selected from the group consisting of sorbitan esters, ethoxylated sorbitan esters, polyalcohols, ethoxylated alky phenols, amine derivatives, amide derivatives, alkylpolyglucosides, ethyleneoxide-propylene-oxide copolymers, thiols or derivatives thereof, poloxamers, pegylated (ethoxylated) fatty acid esters, propoxylated fatty acid esters, mixed ethoxylated/propoxylated fatty acid esters, pegylated (ethoxylated) fatty acid triglycerides, propoxylated fatty acid triglycerides, mixed ethoxylated/propoxylated fatty acid triglycerides, pegylated (ethoxylated) hydroxy substituted fatty acid triglycerides, propoxylated hydroxy substituted fatty acid triglycerides, mixed ethoxylated/propoxylated hydroxy substituted fatty acid triglycerides, wherein said fatty acids are optionally unsaturated, polysorbates, sugar ester, lecithin, bile salts, albumin, alcohols, and mixtures thereof.

In a further embodiment, the surface-active agent is selected from the group consisting of ethoxylated castor oil (polyoxyethylene castor oil); RO 40; BY 140; PEG Castor oil; PEG-10 Castor oil, PEG-100 Castor oil, PEG-1 Castor oil, PEG-15 Castor oil, PEG-2 Castor oil, PEG-20 Castor oil, PEG-200 Castor oil, PEG-25 Castor oil, PEG-26 Castor oil, PEG-3 Castor oil, PEG-30 Castor oil, PEG-33 Castor oil, PEG-35 Castor oil, PEG-36 Castor oil, PEG-4 Castor oil, PEG-40 Castor oil, PEG-5 Castor oil, PEG-50 Castor oil, PEG-54 Castor oil, PEG-55 Castor oil, PEG-60 Castor oil, PEG-8 Castor oil, PEG-9 Castor oil, polyethoxylated castor oil, polyethylene glycol (100) castor oil, polyethylene glycol (11) castor oil, polyethylene glycol (15) castor oil, polyethylene glycol (25) castor oil, polyethylene glycol (26) castor oil, polyethylene glycol (3) castor oil, polyethylene glycol (30) castor oil, polyethylene glycol (33) castor oil, polyethylene glycol (35) castor oil, polyethylene glycol (5) castor oil, polyethylene glycol (50) castor oil, polyethylene glycol (54) castor oil, polyethylene glycol (55) castor oil, polyethylene glycol (60) castor oil, polyethylene glycol 1000 castor oil, polyethylene glycol 1800 castor oil, polyethylene glycol 200 castor oil, polyethylene glycol 2000 castor oil, polyethylene glycol 400 castor oil, polyethylene glycol 450 castor oil, polyethylene glycol 500 castor oil, polyoxyethylene (10) castor oil, polyoxyethylene (100) castor oil, polyoxyethylene (11) castor oil, polyoxyethylene (15) castor oil, polyoxyethylene (2) castor oil, polyoxyethylene (20) castor oil, polyoxyethylene (200) castor oil, polyoxyethylene (25) castor oil, polyoxyethylene (26) castor oil, polyoxyethylene (3) castor oil, polyoxyethylene (30) castor oil, polyoxyethylene (33) castor oil, polyoxyethylene (35) castor oil, polyoxyethylene (36) castor oil, polyoxyethylene (4) castor oil, polyoxyethylene (40) castor oil, Polyoxyethylene (5) castor oil, polyoxyethylene (50) castor oil, polyoxyethylene (54) castor oil, polyoxyethylene (55) castor oil, polyoxyethylene (60) castor oil, polyoxyethylene (8) castor oil, polyoxyethylene (9) castor oil, and combinations thereof.

In an embodiment, the present invention provides for a concentrate composition for solubilizing, dispersing, or emulsifying a water-insoluble or poorly water-soluble active ingredient in an aqueous carrier, said concentrate composition, comprising:
a. a water-insoluble active or poorly water-soluble active ingredient having an HLB value of zero to about 7,
b. a lipophilic component having an HLB value of zero to about 7, and
c. a surface-active agent having an HLB value from about 10 to about 13, wherein said concentrate has a HLB value from about 7 to about 10, and said concentrate is capable of providing a visibly clear aqueous composition.

In a further embodiment, the water-insoluble or poorly water-soluble active ingredient comprises from about 0.01% to about 80%, or from about 0.1% to about 50%, or from about 0.5% to about 25%, or from about 1% to about 20% by weight of the concentrate.

In a further embodiment, the lipophilic component comprises from about 0.1% to about 99.9%, or from about 1% to about 80%, or from about 5% to about 50%, or from about 10% to about 40%, or from about 10% to about 25% by weight of the concentrate.

In a further embodiment, the surface active agent comprises from about 0.1% to about 99.9%, or from about 1% to about 80%, or from about 5% to about 50%, or from about 10% to about 40%, or from about 10% to about 25% by weight of the concentrate. In a further embodiment, the particles formed from the dispersion of the concentrate have a particle size distribution with a mode less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the particles formed from the dispersion of the concentrate have a particle size distribution with a mode in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the particles formed from the dispersion of the concentrate have a particle size distribution with a D50 value less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the particles formed from the dispersion of the concentrate have a particle size distribution with a D50 value in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the water-insoluble active or poorly water-soluble ingredient is selected from the group consisting of essential oils (i.e. also known as plant extracts or botanical extracts), pharmaceutical drug actives, entheogenic plants, mushrooms, psychedelic agents, polypeptides and protein, vitamins, fish oil, milk derivatives, fragrances, flavorings, colorings, sweeteners, taste-enhancers, anti-oxidants, and mixtures thereof.

In a further embodiment, the water-insoluble or poorly water-soluble active ingredient is selected from the group consisting of cannabis extract, hemp oil, human breast milk, cannabinoids, natural phytocannabinoids, organic cannabinoids, endocannabinoids, cannabinoid analogs, cannabinoid derivatives, synthetic cannabinoids, cannabinoid receptor agonists, and mixtures thereof.

In a further embodiment, the cannabinoids are selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydrocannabivarin, delta-8-tetrahydrocannabinolic acid (.DELTA8-THCA), delta-8-tetrahydrocannabinol (DELTAS-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC), and mixtures thereof.

In an embodiment, the present invention provides for a visibly clear aqueous composition of a water-insoluble or poorly water-soluble active ingredient, comprising:
a. a water-insoluble or poorly water soluble active ingredient having an HLB value of zero to about 7,
b. a lipophilic component having an HLB value of zero to about 7,
c. a surface-active agent having an HLB value from about 10 to about 13, and
d. water or an aqueous carrier,
   wherein the combination of said water-insoluble active ingredient, said lipophilic component, and said surface-active agent has a HLB value from about 7 to about 10, and wherein said aqueous composition is a visibly clear dispersion.

A further embodiment relates to a visibly clear aqueous composition according to claim 42 wherein the water-insoluble or poorly water soluble active ingredient comprises from about 0.0001% to about 80%, or from about 0.001% to about 50%, or from about 0.001% to about 25%, or from about 0.01% to about 25%, or from about 0.1% to about 25%, or from about 0.1% to about 10% by weight of the visibly clear aqueous composition.

In a further embodiment, the lipophilic component comprises from about 0.001% to about 25%, or from about 0.01% to about 10%, or from about 0.05% to about 5%, or from about 0.1% to about 2%, or from about 0.1% to about 1% by weight of the visibly clear aqueous composition.

In a further embodiment, the surface active agent comprises from about 0.001% to about 25%, or from about 0.01% to about 10%, or from about 0.05% to about 5%, or from about 0.1% to about 2%, or from about 0.1% to about 1% by weight of the visibly clear aqueous composition.

In a further embodiment, the combination of the water-insoluble or poorly water soluble active ingredient, lipophilic component, and surface-active agent comprise from about 0.002% to about 25%, or from about 0.01% to about 10%, or from about 0.05% to about 5%, or from about 0.1% to about 2%, or from about 0.1% to about 1% by weight of the visibly clear aqueous composition.

In a further embodiment, the combination of the water-insoluble or poorly water soluble active ingredient, lipophilic component, and surface-active agent comprise a concentration from about 20 mg/L to about 250 g/L, or from about 50 mg/L to about 100 g/L, or from about 0.5 g/L to about 10 g/L, or from about 0.75 g/L to about 1.25 g/L in the water or aqueous carrier.

In a further embodiment, the water or aqueous carrier comprises the remainder of the composition by weight.

In a further embodiment, the dispersion comprises particles having a distribution of particle sizes, said distribution having a mode less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the dispersion comprises particles having a distribution of particle sizes, said distribution having a mode in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the dispersion comprises particles having a distribution of particle sizes, said distribution having a D50 value less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

In a further embodiment, the dispersion comprises particles having a distribution of particle sizes, said distribution having a D50 value of the particle size distribution is in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

In a further embodiment, the visibly clear aqueous composition further comprises a water-soluble compound selected from the group consisting of water-soluble plant extracts, pharmaceutical drug actives, vitamins, fragrances, flavorings, colorings, sweeteners, taste-enhancers, anti-oxidants, and mixtures thereof.

In a further embodiment, the water-soluble compound is selected from the group consisting of aloe vera extract, green tea extract, stevia leaf extract, and mixtures thereof.

In a further embodiment, the visibly clear aqueous composition is capable of increasing permeation of the water-insoluble active ingredient through the mucosal or epithelial membrane such as the skin, oral-mucosa, or nasal mucosa by at least about 10% compared to a control composition.

In a further embodiment, the visibly clear aqueous composition is capable of bypassing the first pass metabolism of the water-insoluble active ingredient in a subject (by targeting the lymphatic pathway or by bypassing the oral route of absorption).

In a further embodiment, the visibly clear aqueous composition is capable of extending the release of the water-insoluble active ingredient by at least about 10% compared to a control composition.

In a further embodiment, the visibly clear aqueous composition is in the form of a topical composition for the rejuvenation or treatment of skin, e.g. human skin, in the form of an ointment, a cream, an emulsion, a lotion, a paste, an unguent, a gel or a sunscreen.

In a further embodiment, the visibly clear aqueous composition further comprises a hydroxy acid or hyaluronic acid.

In a further embodiment, the hydroxy acid is an alpha hydroxy acid selected from the group consisting of glycolic acid, citric acid, lactic acid, malic acid, tartaric acid, and mandelic acid.

In a further embodiment, the hydroxy acid is from about 0.1% to about 10% of the composition.

A further embodiment relates to method for making a visibly clear aqueous composition comprising the steps of:
1) combining the lipophilic component(s) and the surface-active agent(s), optionally with mixing and further optionally with the input of sonic energy, to make a preconcentrate,
2) adding the water-insoluble or poorly water-soluble active ingredient(s) to the preconcentrate of step 1), optionally with mixing, to make a concentrate, and
3) adding the concentrate of step 1) to an aqueous system or desired carrier, optionally with mixing, to make the final composition.

A further embodiment relates to a method comprising heating at one or more of steps 1), 2), or 3) to a range of about 40° C. to about 100° C.

A further embodiment relates to a method comprising applying sonic energy to the mixture at step 1) having a frequency from about 180-990 Hz.

These and other embodiments of the present invention will become apparent from the disclosure herein.

### BRIEF DESCRIPTION OF THE DRAWINGS

FIG. 1 presents the results of a solubilization study (run in accordance with USP standards: see Stage 6 Harmonization, Official Dec. 1, 2011<711> Dissolution) comparing the release of cannabidiol from an aqueous formulation of the present invention made using concentrate of Formulation A, versus two-marketed formulations, into a simulated gastrointestinal medium.

FIG. 2 presents the results of a solubilization study (run in accordance with USP standards: see Stage 6 Harmonization, Official Dec. 1, 2011<711> Dissolution) comparing the release of cannabidiol from two concentrate compositions of the present invention (Formulations B and C), into a simulated gastrointestinal medium.

FIG. 3 presents the results of a solubilization study conducted in the presence of bile salts (at concentrations of 10 mM (millimolar), 15, mM, 20 mM, and 40 mM) into a simulated gastrointestinal medium, comparing the solubility of probucol, a water-insoluble active ingredient, from a concentrate composition of the present invention (Formulation B) versus a simple aqueous formulation of probucol, as a control (without the lipophilic components and surface active agents).

FIG. 4 presents the results of a permeation study in a simulated human intestinal cell line model, comparing the permeation of probucol, a water-insoluble active ingredient, from a concentrate composition of the present invention (Formulation B) versus a simple aqueous formulation of probucol, as a control (without the lipophilic components and surface-active agents). The permeation from both a trans-cellular and a para-cellular pathway are simulated and compared.

FIG. 5 presents a representative population trace of the hydrodynamic radius for an exemplary visibly clear aqueous composition measured by dynamic light scattering (DLS) measurements. The X-axis is shown on a logarithmic scale.

FIG. 6 presents diagram of the biopharmaceutics classification system. This representation is based upon the published work of Amidon, G. L., et al., A Theoretical Basis for a Biopharmaceutic Drug Classification: The Correlation of in Vitro Drug Product Dissolution and in Vivo Bioavailability, Pharm Res 12, 413-420 (1995), and FDA guidelines relating to pharmacokinetic parameters for drug actives.

### DETAILED DESCRIPTION OF THE INVENTION

The present invention provides a technology platform to improve water solubility and permeability of active ingredients through oral, topical, and mucosal routes. The present invention incorporates the active ingredients in the form of single chemical compounds, mixtures of actives, or full plant botanical extracts or powders in various lipid carriers for formulation into an aqueous carrier, to provide a visually clear composition. The varying hydrophilicity and lipophilicity of the lipid carriers are adjusted to provide the desired balance oil-water miscibility. In further embodiments, the compositions can be prepared utilizing sonic wave energy, as for example from a sonicator, to aid dispersion. The optional sonic energy input can be used to facilitate the preparation of a preconcentrate comprising the lipophilic component and surface active agent.

### Definitions

As used herein, the following terms and abbreviations have the indicated meanings unless expressly stated to the contrary.

The abbreviation “CBD” as used herein means cannabidiol, which is a cannabinoid and a major component of cannabis extracts.

The term “HLB” is well-known in the art and is an abbreviation for “hydrophile-lipophile balance” or ‘hydrophilic-lipophilic balance”, and is an empirical expression for the relationship of the hydrophilic (“water-loving”) and hydrophobic (“water-hating”) groups of a surfactant or material. The “HLB” denotes a surface tension value to solubilize the water-insoluble or poorly water soluble active. The “HLB” scale ranges from zero to 20.

Term abbreviation “mM” as used herein means millimolar concentration.

Term abbreviation “nm” as used herein means nanometers.

Term abbreviation “mg/ml” or “mg/I” used herein means milligrams per milliliters or milligrams per liters respectively.

Term abbreviation “% w/w” as used herein means percentage weight by weight.

Term abbreviation “USP” as used herein means United States Pharmacopeia.

The term “thermodynamically stable” means that the oil droplets will not coalesce and will remain dispersed evenly throughout an aqueous phase resulting in the solubilization of an active for an indefinite time within acceptable temperature and pressure changes.

The term “emulsion” is a multi-particulate system of oil droplets dispersed in an aqueous carrier, typically ranging in size from about 250 nanometers and above. This system is not thermodynamically stable and the oil droplets tend to eventually coalesce and separate from the aqueous carrier. The term “emulsion” can also be used in a general sense and is not intended to define the size of the particles in a visibly clear aqueous solution. A definitive particle size should not be inferred based on the term “emulsion” or, alternatively, “dispersion”, and should only be taken if expressly recited. The term “emulsion” can be broadly used to distinguish from “mico-emulsion” and “nano-emulsion” as described herein.

The term “micro-emulsion” is a multi-particulate system of oil droplets dispersed in an aqueous carrier typically ranging in size from about 10 to about 250 nanometers and is thermodynamically stable.

The term “nano-emulsion” is a multi-particulate system of oil droplets dispersed in an aqueous carrier typically ranging in size from about 5 to about 250 nanometers and is not thermodynamically stable and the oil droplets tend to eventually coalesce and separate from the aqueous carrier.

The key differences between microemulsions and emulsions or nano-emulsions is the thermodynamic stability that prevents the micro-emulsions to phase separate upon storage. Emulsions are generally defined as having particles of about 250 nm and above. Emulsions are generally thermodynamically unstable. Microemulsions are generally defined as having particles of about 10-250 nm. Microemulsions are generally thermodynamically stable. Nanoemulsions are generally defined as having particles of about 5 to 250 nm. Nanoemulsions are generally thermodynamically unstable.

The following Table A summarizes the characteristics of emulsions, microemulsions, and nanoemulsions.

TABLE A
Comparison of Emulsion Characteristics

| | Approximate | |
|---|---|---|
| | Size Range | Stability |
| Emulsions | 250 nm and above | Thermodynamically Unstable |
| Microemulsions | 10-250 nm | Thermodynamically Stable |
| Nanoemulsions | 5-250 nm | Thermodynamically Unstable |

In the present invention, the visibly clear aqueous compositions are generally found to have particle sizes below 250 nm, and in some instances even around or below about 10 nm; however, these systems are found to have good stability based on DLS data. In some instances, stable particles may have particle sizes of about 8 nm. It can be appreciated that the exact size ranges for “microemulsions” and “nanoemulsions” are not concretely known and can vary greatly depending on the composition of the emulsions. Therefore, it may be important to verify stability by visual inspection and DLS measurements, or another appropriate measurement to analyze particle size, performed over the course of a period of time in order to analyze stability over said period of time before making an assumption on the stability a priori based only upon the particle size.

The term “pharmaceutically acceptable” is used herein with respect to the compositions, in other words the formulations, of the present invention. The pharmaceutical compositions of the present invention can comprise a therapeutically effective amount of active ingredient such as a cannabinoid and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17th edition, edited by Alfonso R. Gennaro, Mack Publishing Company, Easton, Pa., 17th edition, 1985.

The term “subject” means a human patient or animal in need of treatment or intervention for pain or pruritus, particularly neuropathic or chronic inflammatory pain and/or pruritus.

The abbreviation “THC” means tetrahydrocannabinol or derivatives thereof, which is a cannabinoid and a major component of cannabis extracts.

The term “therapeutically effective” means an amount of active ingredient needed to provide a meaningful or demonstrable benefit, as understood by medical practitioners, to a subject, such as a human patient or animal, in need of treatment. The demonstration of a benefit can also include those provided by models, including but not limited to in vitro models, in vivo models, and animal models.

The terms “treat,” “treating” or “treatment,” as used herein, include alleviating, abating or ameliorating the condition, e.g. pain or pruritus, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The term “ethoxylated” as used herein refers to a molecule or chemical group being substituted with one or more groups originating from ethylene glycol and/or ethylene oxide, or their equivalents. The term “ethoxy” encompasses both an individual group originating from ethylene oxide or ethylene glycol, or a polymeric group originating from one or more ethylene oxide and/or ethylene glycol molecules. An “ethoxylated” molecule or chemical group comprises at least one “ethoxy” group. A molecule or chemical group may be ethoxylated at any viable position, including for example at an alcohol or carboxylic acid functional group. The terms “ethoxy” and “ethoxylated” also encompass the terms polyethylene glycol (PEG), pegylated, polyethylene oxide (PEO), and polyoxoethylene, and any other synonymous terms known or understood by a person of skill in the art.

In some embodiments, an ethoxy group may comprise 1 to about 1000, or 1 to about 900, or 1 to about 800, or 1 to about 700, or 1 to about 600, or 1 to about 500, or 1 to about 400, or 1 to about 300, or 1 to about 200, or 1 to about 100, or 1 to about 90, or 1 to about 80, or 1 to about 70, or 1 to about 60, or 1 to about 50, or 1 to about 45, or 1 to about 40, or 1 to about 35, or 1 to about 30, or 1 to about 25, or 1 to about 20, or 1 to about 15, or 1 to about 10, or 1 to about 5 units originating from ethylene glycol or ethylene oxide, inclusive of any sub-ranges within any of those ranges. In some embodiments, an ethoxy group may comprise about 1000, or about 950, or about 900, or about 850, or about 800, or about 750, or about 700, or about 650, or about 600, or about 550, or about 500, or about 450, or about 400, or about 350, or about 300, or about 250, or about 200, or about 150, or about 100, or about 95, or about 90, or about 85, or about 80, or about 75, or about 70, or about 65, or about 60, or about 55, or about 50, or about 45, or about 40, or about 35, or about 30, or about 25, or about 20, or about 18, or about 16, or about 14, or about 12, or about 10, or about 8, or about 6, or about 4, or about 2, or 1 units originating from ethylene glycol or ethylene oxide.

The term “propoxylated” as used herein refers to a molecule or chemical group being substituted with one or more groups originating from propylene glycol and/or propylene oxide, or their equivalents. The term “propoxy” encompasses both an individual group originating from propylene oxide or propylene glycol, or a polymeric group originating from one or more propylene oxide and/or propylene glycol molecules. A “propoxylated” molecule or chemical group comprises at least one “propoxy” group. A molecule or chemical group may be propoxylated at any viable position, including for example at an alcohol or carboxylic acid functional group. The terms “propoxy” and “propoxylated” also encompass the terms polypropylene glycol (PPG), polypropylene oxide, and any other synonymous terms known or understood by a person of skill in the art.

In some embodiments, a propoxy group may comprise 1 to about 1000, or 1 to about 900, or 1 to about 800, or 1 to about 700, or 1 to about 600, or 1 to about 500, or 1 to about 400, or 1 to about 300, or 1 to about 200, or 1 to about 100, or 1 to about 90, or 1 to about 80, or 1 to about 70, or 1 to about 60, or 1 to about 50, or 1 to about 45, or 1 to about 40, or 1 to about 35, or 1 to about 30, or 1 to about 25, or 1 to about 20, or 1 to about 15, or 1 to about 10, or 1 to about 5 units originating from propylene glycol or propylene oxide, inclusive of any sub-ranges within any of those ranges. In some embodiments, a propoxy group may comprise about 1000, or about 950, or about 900, or about 850, or about 800, or about 750, or about 700, or about 650, or about 600, or about 550, or about 500, or about 450, or about 400, or about 350, or about 300, or about 250, or about 200, or about 150, or about 100, or about 95, or about 90, or about 85, or about 80, or about 75, or about 70, or about 65, or about 60, or about 55, or about 50, or about 45, or about 40, or about 35, or about 30, or about 25, or about 20, or about 18, or about 16, or about 14, or about 12, or about 10, or about 8, or about 6, or about 4, or about 2, or 1 units originating from propylene glycol or propylene oxide.

The term “mixed ethoxylated/propoxylated” refers to a molecule or chemical group being substituted with one or more groups that are a random, alternating, and/or block copolymer originating from two or more of ethylene glycol, ethylene oxide, propylene glycol, and propylene oxide or their equivalent moieties. The term “mixed ethoxy/propoxy” encompasses a group that is a random, alternating, and/or block copolymer originating from two or more of ethylene glycol, ethylene oxide, propylene glycol, and propylene oxide. A “mixed ethoxylated/propyxylated” molecule of chemical group comprises at least one “mixed ethoxy/propyxy” group. A molecule or chemical group may be “mixed ethoxylated/propyxylated” at any viable position, including for example at an alcohol or carboxylic acid, functional group.

In some embodiments, a mixed ethoxy/propoxy group may comprise 2 to about 1000, or 2 to about 900, or 2 to about 800, or 2 to about 700, or 2 to about 600, or 2 to about 500, or 2 to about 400, or 2 to about 300, or 2 to about 200, or 2 to about 100, or 2 to about 90, or 2 to about 80, or 2 to about 70, or 2 to about 60, or 2 to about 50, or 2 to about 45, or 2 to about 40, or 2 to about 35, or 2 to about 30, or 2 to about 25, or 2 to about 20, or 2 to about 15, or 2 to about 10, or 2 to about 5 units originating from two or more of ethylene glycol, ethylene oxide, propylene glycol or propylene oxide, inclusive of any sub-ranges within any of those ranges. In some embodiments, a propoxy group may comprise about 1000, or about 950, or about 900, or about 850, or about 800, or about 750, or about 700, or about 650, or about 600, or about 550, or about 500, or about 450, or about 400, or about 350, or about 300, or about 250, or about 200, or about 150, or about 100, or about 95, or about 90, or about 85, or about 80, or about 75, or about 70, or about 65, or about 60, or about 55, or about 50, or about 45, or about 40, or about 35, or about 30, or about 25, or about 20, or about 18, or about 16, or about 14, or about 12, or about 10, or about 8, or about 6, or about 4, or about 2 units originating from two or more of ethylene glycol, ethylene oxide, propylene glycol or propylene oxide.

The methods of treatment using the compositions of the present invention, in various embodiments also include the use of the active ingredients or the compositions in the manufacture of a medicament for the desired treatment.

The terms “visually clear” or “visibly clear” as used herein with respect to the compositions of the present invention, and in particular the aqueous compositions of the present invention means that the compositions to the unaided eye of the average observer appear clear, transparent, or water white, or without turbidity, milkiness, opacity, cloudiness, or noticeable particles. It is recognized that such a visibly clear composition is not necessarily a true solution, but can be a dispersion, emulsion, or other type of complex composition in which particles are not visible to the unaided eye of the average observer. In other embodiments, the visibly clear aqueous compositions of the present invention have a clarity when measured by dynamic light scattering (DLS) indicating that any particles present in the composition have a particle size of less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

The term “particle size” as used herein refers to the size of solubilized, dispersed, or emulsified concentrate or preconcentrate particles formed in an aqueous carrier and/or aqueous solution. The particle size may be determined by any appropriate technique, including dynamic light scattering (DLS). Dynamic light scattering (DLS) is a physical technique that typically uses a polarized monochromatic light source, such as a laser source. The particles in the sample scatter the light which is typically sent through a second polarizer and projected for collection and analysis. The particles normally exist in a distribution of sizes and the particle size can be determined or reported in various ways as described herein. The term “particle size”, unless otherwise stated, herein refers to the mode or peak value of a number (population) hydrodynamic radius distribution determined by DLS. These particle sizes can be reported or described by various metrics, including particle size (mode), D50, and D90 as described herein, including in the section “particle size and transparency”.

The terms “water-insoluble active” and “poorly water-soluble active” as used herein mean an active ingredient as described below that has a solubility of less than about 10 mg/ml of water at 25° C., or less than about 1 mg/ml of water at 25° C., or less than about 0.1 mg/I of water at 25° C. It is recognized that most substances have some solubility in water, even if extremely small. However, this definition is intended to define that the solubility of the substance is less than the indicated value to distinguish it from materials having water solubility greater or equal to the indicated values. Furthermore, these water-insoluble actives and poorly water-soluble actives can be further defined herein as having an HLB value of zero to about 7. The term “HLB” is well-known in the art and is an abbreviation for “hydrophile-lipophile balance” or ‘hydrophilic-lipophilic balance”, and is an empirical expression for the relationship of the hydrophilic (“water-loving”) and hydrophobic (“water-hating”) groups of a surfactant or material. The scale ranges from zero to 20.

Furthermore, the solubility and permeability of the active ingredients can be characterized using the Biopharmaceutical (also known as the Biopharmaceutics) Classification System (BCS). The BCS is the standardized system utilized in the pharmaceutical and nutraceutical industries, as defined by the US Food and Drug Administration, to classify the solubility and permeation characteristics of various active ingredients. These properties are useful, for example, for bioavailability and bioequivalence studies.

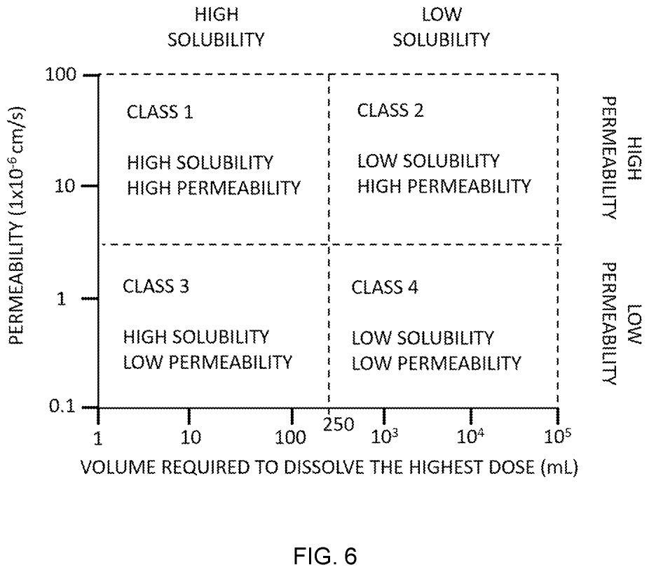

Biopharmaceutical Classification System was originally developed in 1995, by Amidon et al. See, Amidon, G. L., et al., A Theoretical Basis for a Biopharmaceutic Drug Classification: The Correlation of in Vitro Drug Product Dissolution and in Vivo Bioavailability. Pharm Res 12, 413-420 (1995). The system can be described as a scientific framework for classifying a drug substance based, aqueous solubility, intestinal permeability, and dissolution rate. The system was developed to provide a scientific approach for predicting the in vivo pharmacokinetics of an oral drug product. The Biopharmaceutical Classification System is exemplified in FIG. 6, which is based on the work of Amidon et al.

Solubility refers to the ability of a compound to be dissolved in water, and permeability refers to the ability of a compound to pass through the gut membrane into the bloodstream. According to the Biopharmaceutical Classification System, drug actives are defined as falling into one of four classes, defined as follows: Class 1 compounds have high solubility and high permeability. Class 2 compounds have low solubility and high permeability. Class 3 compounds have high solubility and low permeability. Class 4 compounds have low solubility and low permeability.

Examples of drug substances that fall into the four classes include:
Class 1: metoprolol, propranolol, paracetemol
Class 2: nifedipine, naproxen, aceclofenac
Class 3: cimetidine, metformin
Class 4: taxol, clorthiazole, bifonazole

Based on publicly available information it is reported that:

Solubility class boundaries are based on the highest dose strength of an immediate release drug product, where a drug is considered highly soluble when the highest dose strength is soluble in 250 ml or less of aqueous media over the pH range of 1 to 7.5. The volume estimate of 250 ml is derived from typical bioequivalence study protocols that prescribe administration of a drug product to fasting human volunteers with a glass of water.

Permeability class boundaries are based indirectly on the extent of absorption of a drug substance in humans and directly on the measurement of rates of mass transfer across human intestinal membrane. Alternatively, non-human systems capable of predicting drug absorption in humans can be used (such as in-vitro culture methods). A drug substance is considered highly permeable when the extent of absorption in humans is determined to be 90% or more of the administered dose based on a mass-balance determination or in comparison to an intravenous dose.

For dissolution class boundaries, an immediate release product is considered rapidly dissolving when no less than 85% of the labeled amount of the drug substance dissolves within 15 minutes using a USP Dissolution Apparatus 1 at 100 RPM or an Apparatus 2 at 50 RPM in a volume of 900 ml or less in the following media: 0.1 M HCl or simulated gastric fluid or pH 4.5 buffer and pH 6.8 buffer or simulated intestinal fluid.

The present invention is useful for enhancing the solubility and/or permeability of compounds in classes 2, 3, and 4, although there can be instances where the invention can be useful for compounds in class 1. The present invention can also enhance the solubility and/or permeability of multiple compounds from multiple classes simultaneously. It is contemplated that the Biopharmaceutical Classification System is presented here as a guide for defining water solubility of the active compounds to be formulated according to the present invention, and not intended to limit the present invention.

Many botanicals, pharmaceuticals, and nutraceutical compounds fall into Class 4, which means these actives can benefit immensely from the application of the present invention. However, even compounds in Class 1, can benefit from the present in invention from advantages such as enhancing bioavailability, e.g., by bypassing hepatic first-pass metabolism, inhibiting p-glycoprotein efflux, and inhibition of CIP-450. The present invention is useful not only for the improvements it provides for solubility and permeability of active ingredients, but also in its ability to enhance multiple classes of compounds simultaneously.

### Compositions of the Present Invention

As discussed above, many currently-available compositions containing water-insoluble or poorly water-soluble active ingredients are often formulated as single-phase oil-based compositions, turbid emulsions, or micro-encapsulated systems with limited stability. These compositions are less than ideal. Furthermore, the formulation challenges are compounded for the delivery of cannabinoid compounds and cannabis extracts. For example, existing cannabinoid products for topical, transdermal, or mucosal applications are using CBD dispersed in oil, which has low permeability, or aqueous CBD isolate formulations which offer an inferior benefit compared to full-spectrum CBD.

The present invention overcomes these solubilization and delivery challenges for water-insoluble or poorly water-soluble active ingredients, and particularly for cannabinoids and cannabis extracts.

The present invention provides a solubilization platform for water-insoluble or poorly water-soluble active ingredients. The platform allows the creation of solutions and products with attributes not readily achieved with water-insoluble or poorly water-soluble materials. The desirable attributes unlocked by the present invention, can include one or more of the following:
1). visually clear solutions,
2). close to 100% or 100% solubilization or dispersion of the active ingredients,
3). long shelf life stability,
4). rapid onset of action for the active ingredients,
5). consistent onset of action for the active ingredients,
6). consistent potency for the active ingredients in each product dose and precise batch to batch consistency in the manufacturing process,
7). consistent active ingredient dosing,
8). bypass of hepatic first pass metabolism of the active ingredients,
9). increased permeation of the active ingredients through the skin and mucosal membranes for topical, transdermal, and intranasal applications,
10). the ability to solubilize complete organic, raw plant extracts, such as full-spectrum hemp or cannabis crude oil,
11). the ability to deliver both water-insoluble, and if also desired, water-soluble actives from the same composition, and
12). improved bioavailability for the active ingredients.

Not only do these attributes improve the quality of existing product applications, but the present invention, also opens the door to a new dimension of innovative product applications not readily achievable before. The present invention, i.e. the delivery platform, provides solutions and products not previously possible for water-insoluble and poorly water-soluble materials, such as cannabinoids and cannabis extract.

The aesthetic breakthroughs of present invention include the creation of clear solutions of botanical extracts in water-based beverages, as well as non-greasy topical and transdermal products, for better consumer appeal. Current products and methods for creating water-soluble cannabinoids typically produce cloudy, milky solutions.

The technology of the present invention includes the creation of water-soluble and highly permeable product compositions for various dosage forms including oral, topical and transdermal applications. The present invention can provide for an effective platform to deliver therapeutic molecules and/or botanical extracts, such as cannabinoids, in a wide array of product applications. Possible product categories include, but are not limited to, pharmaceuticals, nutraceuticals, CBD products, marijuana products (e.g., medical and recreational), animal health products, and cosmetics. The possible product applications in the oral route of administration include oral liquids, beverage additives, flavored or unflavored beverages, sublingual liquids, buccal liquids, oral-mucosal sprays or mists, capsules, tablets, and dry powders. The product applications for mucosal delivery include intranasal liquids or sprays, topical products, transdermal patches, suppositories, capsules, tablets and dry powders. The high bioavailability of the active ingredients in the products of the present invention, can reduce the required dose quantities by 50% to 95%. These lower requirements can reduce manufacturing costs for producers by decreasing the amount of required raw materials, while also providing a superior experience for consumers with reduced risk of adverse side effects, due to lower dosing requirements.

### Preconcentrate

The preconcentrate comprises a lipophilic component having a hydrophilic-lipophilic balance (HLB) value of zero to about 7, and a surface-active agent having an HLB value from about 10 to about 13. The preconcentrate composition is useful for solubilizing, dispersing, and/or emulsifying a water insoluble or poorly water-soluble active ingredient in an aqueous carrier. The preconcentrate does not comprise the water insoluble or poorly water-soluble active ingredient. Upon addition of the water insoluble or poorly water-soluble active ingredient, the composition is referred to as a concentrate.

The lipophilic component of the preconcentrate may comprise one or more different components and the term “lipophilic component” used in the singular is intended to include one or more lipophilic components. The surface-active agent of the preconcentrate may comprise one or more different components and the term “surface active agent” used in the singular is intended to include one or more surface active agents. The preconcentrate, by weight percent, may be comprised of between 0.1-99.9% lipophilic component and 0.1-99.9% surface active agent, realizing that if one component accounts for a certain weight percentage X of the lipophilic component, the surface active agent would be equal or less than (100−X)%. In some embodiments, the mass percentages of the lipophilic component and surface-active agent do not necessarily need to add to 100%, as further additives, excipients, stabilizers, impurities, moisture, adventitious materials, or other components may be present in the preconcentrate. If overlapping weight percentage ranges are provided that could potentially add to greater than 100%, it can be appreciated that a person of skill in the art would be readily able to determine which values for each component are possible.

### Concentrate

The concentrate comprises a lipophilic component, a surface-active agent, and one or more water insoluble or poorly water-soluble active ingredients. Stated in another way, the concentrate comprises the preconcentrate and one or more water insoluble or poorly water-soluble active ingredients. With the one or more active ingredients added, the concentrate has different weight percentage values relative to the corresponding preconcentrate used to produce the concentrate. If A is the weight percentage of the active ingredient and X is the weight percentage of the lipophilic component, the weight percentage of the surface active agent would be less than or equal to (100−X−A)%. Likewise, if A is the weight percentage of the active ingredient and Y is the weight percentage of the surface active agent, the weight percentage of the lipophilic component would be less than or equal to (100−Y−A)%. Likewise, if X is the weight percentage of the lipophilic component and Y is the weight percentage of the surface active agent, the weight percentage of the active ingredient would be less than or equal to (100−X−Y)%. In some embodiments, the mass percentages of the lipophilic component, surface active agent, and water insoluble or poorly water soluble active ingredient(s) do not necessarily need to add to 100%, as further additives, excipients, stabilizers, impurities, moisture, adventitious materials, or other components may be present in the concentrate. If overlapping weight percentage ranges are provided that could potentially add to greater than 100%, it can be appreciated that a person of skill in the art would be readily able to determine which values for each component are possible.

### Visibly Clear Aqueous Composition

The visibly clear aqueous composition is produced by combining the concentrate, comprising a lipophilic component, a surface active agent, and one or more water insoluble or poorly water soluble active ingredients, with water or an aqueous carrier. The result is a dispersion, suspension, or emulsion of the concentrate in water or the aqueous carrier yielding a visibly clear aqueous composition. In some embodiments, the resultant dispersion, suspension, or emulsion has a particle size sufficiently small such that the aqueous composition is visibly clear. Various quantitative descriptions of the visibly clear aqueous composition are possible.

The particle size of the dispersion, suspension, or emulsion of the concentrate in the composition may be determined as disclosed herein or in any other manner understood by a person skilled in the art. The weight percentage of each component, including water or the aqueous carrier, may be used. The concentration in mg/mL, or in any other appropriate unit such as molarity, may be used to describe the total concentration of the active ingredient in the composition. Concentrations could also be used to describe any other component of the composition, including the lipophilic component, the surface active agent, and any optional additives, excipients, stabilizers, or other components.

The visibly clear aqueous composition may be defined by several quantitative values with respect to its components. In some embodiments, the visibly clear aqueous composition is defined by the mass percentage of the lipophilic component. In some embodiments, the lipophilic component comprises from about 0.001% to about 25%, or from about 0.01% to about 10%, or from about 0.05% to about 5%, or from about 0.1% to about 2%, or from about 0.1% to about 1% by weight of the visibly clear aqueous composition. These ranges are inclusive of any values inbetween such as about 0.001%, or about 0.01%, or about 0.1%, or about 1%, or about 10%, Any ranges encompassing these values are contemplated.

In some embodiments, the visibly clear aqueous composition is defined by the mass percentage of the surface active agent. In some embodiments, the surface active agent comprises from about 0.001% to about 25%, or from about 0.01% to about 10%, or from about 0.05% to about 5%, or from about 0.1% to about 2%, or from about 0.1% to about 1% by weight of the visibly clear aqueous composition. These ranges are inclusive of any values inbetween such as about 0.001%, or about 0.01%, or about 0.1%, or about 1%, or about 10%, Any ranges encompassing these values are contemplated.

In some embodiments, the visibly clear aqueous composition is defined by the mass percentage of the water insoluble or poorly water soluble active ingredient. In some embodiments, the water-insoluble or poorly water soluble active ingredient comprises from about 0.0001% to about 80%, or from about 0.001% to about 50%, or from about 0.001% to about 25%, or from about 0.01% to about 25%, or from about 0.1% to about 25%, or from about 0.1% to about 10% by weight of the visibly clear aqueous composition. These ranges are inclusive of any values inbetween such as about 0.0001%, or about 0.001%, or about 0.01%, or about 0.1%, or about 1%, or about 10%, Any ranges encompassing these values are contemplated.

In some embodiments, the visibly clear aqueous composition may be defined by mass percentage of the combination of the water-insoluble or poorly water soluble active ingredient, lipophilic component, and surface active agent (i.e. the concentrate). In some embodiments, the concentrate comprises from about 0.002% to about 25%, or from about 0.01% to about 10%, or from about 0.05% to about 5%, or from about 0.1% to about 2%, or from about 0.1% to about 1% by weight of the visibly clear aqueous composition. These ranges are inclusive of any values inbetween such as about 0.0021%, or about 0.005%, or about 0.009%, or about 0.01%, or about 0.1%, or about 1%, or about 10%, Any ranges encompassing these values are contemplated.

In some embodiments, a concentration of the water-insoluble or poorly water soluble active ingredient, lipophilic component, and surface active agent (i.e. the concentrate) in water or an aqueous carrier may be used to define the visibly clear aqueous composition. The concentration may be provided in units of (mass of concentrate)/(volume of water or aqueous carrier). In some embodiments, the unit is mg/L, or g/L. In some embodiments, the water-insoluble or poorly water soluble active ingredient, lipophilic component, and surface-active agent together (i.e. the concentrate) comprise a concentration from about 20 mg/L to about 250 g/L, or from about 50 mg/L to about 100 g/L, or from about 0.5 g/L to about 10 g/L, or from about 0.75 g/L to about 1.25 g/L in the water or aqueous carrier. These ranges are inclusive of any values inbetween such as about 20 mg/L, or about 50 mg/L, or about 100 mg/L, or about 500 mg/L, or about 750 mg/L, or about 1.25 g/L, or about 1.5 g/L, or about 5 g/L, or about 100 g/L, or about 150 g/L, or about 250 g/L. Any ranges encompassing these values are contemplated.

In some embodiments, a visibly clear aqueous composition having specific or range-defined weights of water insoluble active ingredient(s), lipophilic component(s), and surface-active agent(s) comprises water or aqueous carrier as the remainder of the composition by weight. It can be appreciated that one of ordinary skill in the art can realize that the amount of water or aqueous carrier can be adjusted to complete the formula, i.e. to give a Q.S. to achieve 100% by weight.

### Particle Size and Transparency

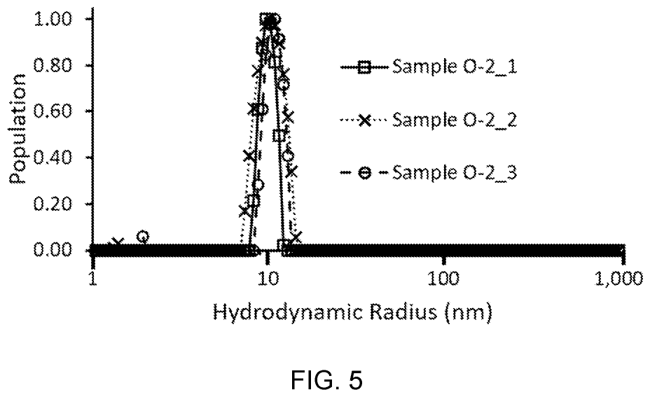

The particle size distribution of the visibly clear aqueous composition of the present invention may be determined by any appropriate experimental technique, including dynamic light scattering (DLS). DLS measures an intensity correlation function for particles in suspension undergoing Brownian motion. From the intensity correlation function, a distribution of the hydrodynamic radius can be obtained. There are different ways to represent this distribution, such as intensity, volume or mass, or number (population). For the present invention, most DLS distributions are number or population distributions (see the exemplary distribution in FIG. 5). The exemplary distribution in FIG. 5 shows predominantly a single peak and is therefore indicative of a monodisperse system. If one or more peaks are obtained, a system is said to be polydisperse. In some embodiments, the visibly transparent aqueous composition is monodisperse. In some embodiments, the visibly transparent aqueous composition is polydisperse.

Various particle size metrics may be determined from the hydrodynamic radius distribution. In both monodisperse and polydisperse systems, the particle size may be determined from the peak value of the hydrodynamic radius distribution (i.e. the mode), from the median of the distribution, or from the mean of the distribution. Distribution widths may also be given to describe a distribution, such as from the full width at half max (FWHM). Integrated areas of the distribution may also be used to determine metrics for particle size. For instance, the distribution can be normalized by total area and then integrated over a range encompassing a certain percentage of the total area. The definite integral may have a lower bound corresponding the smallest particle size in the distribution and may have an upper bound corresponding to the particle size at which the area integrated encompasses a certain percentage of the total area. The percentage of the total area integrated could be any integer or non-integer value from 0.01%-99.99%.

As a convenient convention, the percentage of the total area used to report a particle size may be D50 and/or D90, corresponding an integral of 50% or 90%, respectively, of the total area of the distribution. A D50 value represents a value where 50% of the particles have a particle size smaller than the D50 value, and the other 50% of the particles have a particle size larger than the D50 value. A D90 value represents a value where 90% of the particles have a particle size smaller than the D90 value, and the other 10% of the particles have a particle size larger than the D90 value. These metrics, such as D50 and D90, are calculated starting from the hydrodynamic radius (Rₕ) distribution function. The distribution function is then normalized by the total area to obtain the probability function, p(Rₕ). The below integral can then be evaluated over integral bounds that capture, for instance, 50% of the total area for a D50 calculation, or 90% of the total area for a D90 calculation, where Bₗ is the lower bound and Bᵤ is the upper bound.

∫_{B}_{ₗ}^{B}^{ᵤ}Rₕ×p(Rₕ)dRₕ

The mechanism of light scattering depends upon the particle size. Rayleigh scattering can be used to describe scattering from particles having a size much smaller than the wavelength of light (i.e. less than about 1/10^{th} of the wavelength of light, such as 40 nm particle size for 400 nm light). The intensity of Rayleigh scattering scales as d⁶, where d is the particle diameter. For somewhat larger particles relative to visible light wavelengths (roughly 40 nm to 900 nm), Tyndall scattering may be relevant to determining and/or comparing particle sizes between two different emulsions. Under Tyndall scattering, at a given wavelength, a larger particle has a higher degree of scattering than a smaller particle. Likewise, under either scattering type, a distribution of particles having a larger particle size (such as a larger D50 or D90 value) will exhibit more light scattering than a distribution of particles having a smaller particle size. These scattering mechanisms are relevant to both static and dynamic light scattering. In some embodiments, static light scattering provides a useful description for transparency and for methods to measure particle size. Static light scattering may be determined from an angular intensity distribution. Some useful techniques may include a nephelometer or aerosol photometer, a turbidimeter, and/or an ultramicroscope. Mie theory is a particular example of a theory falling under the Tyndall mechanism that may be used to describe particle sizes of spherical particles. It can be appreciated that any appropriate method to measure or determine the particle size of an emulsion may be performed and used to make scientifically valid comparisons to the particle sizes obtained from DLS. In some embodiments, only some of the particle size metrics may be available from each technique and only comparable metrics should be compared.

Because light scattering theories, where appropriately applied to the particle sizes that they are capable of describing, hold that larger particles exhibit a larger degree of scattering, the maximization of “clarity” or “transparency” of an emulsion or dispersion can be understood as a minimization of light scattering which is associated with a minimization of the particle size. Therefore, in some embodiments, particle size metrics may be important for describing the transparency of visibly clear aqueous solutions or emulsions having a distribution of particle sizes. In some embodiments, particle size metrics may be used to define transparency or visible clarity. Alternatively, particle size metrics may be used to differentiate transparency characteristics of one composition or emulsion from another. For instance, a first composition or emulsion having a smaller particle size than a second composition or emulsion can be considered to have a higher transparency or visible transparency. In some embodiments, the transparency, visible transparency, and/or clarity of a given first composition or emulsion may be superior to a given second composition or emulsion if the particle size of the first composition or emulsion is smaller than the particle size of the second composition or emulsion, according to one or more particle size metrics.

### Unmet Needs Provided by the Present Invention

The present invention provides water-soluble compositions for active ingredients such as cannabinoids that are clear, stable, and have good delivery characteristics. The present invention provides superior product efficacy, minimizes adverse side effects, increases consumer appeal, and reduces production costs. Based on current market assessments, the present invention is a unique platform that can successfully incorporate water-insoluble, poorly water-soluble, or poorly permeable isolated molecules as well as full plant botanical extracts or powders. This feature has opened the door to scientifically delivering organic plants and botanicals in the supplement industry.

The cannabis industry continues to grow rapidly with the global market expected to reach $73.6B by 2027 according to Grandview Research (Legal Marijuana Market Size 2020). See, Legal Marijuana Market Size Worth $73.6 Billion By 2027. (2020, February). Retrieved May 17, 2020, from https://www.grandviewresearch.com/press-release/global-legal-marijuana-market. Within the cannabis market, CBD represents the fastest-growing segment, and many analysts contend that CBD is a larger long-term market opportunity than marijuana due to its potential to augment (if not replace) existing medications and recreational substances like tobacco or alcohol.

Equity Data Science Analytics projects the CBD market in the United States alone will hit $20B in 2024, while the Brightfield Group projects US CBD sales to eclipse $20B as early as 2022. See, Dorbian, I. (2019, Oct. 15). CBD Market Could Reach $20 Billion By 2024, Says New Study. Retrieved Apr. 3, 2020, from https://www.forbes.com/sites/irisdorbian/2019/05/20/cbd-market-could-reach-20-billion-by-2024-says-new-study/. For comparison, the entire US beer market in 2018 was $35B while CBD sales in the United States in 2018 were roughly $500M. See, Kendall, J. (2019, Jan. 14). IRI: US Beer Sales Top $35 Billion in 2018. Retrieved Jun. 1, 2020, from https://www.brewbound.com/news/iri-us-beer-sales-top-35-billion-in-2018.

This growth opportunity is drawing attention from entrepreneurs, established cannabis companies, and Fortune 500 companies. Many firms, historically far afield of the cannabis industry, are taking decisive strides to establish a significant presence in CBD, including mainstream pharmacies and national grocery chains. Additionally, thousands of specialty CBD stores have opened across the country, and tens of thousands of small convenience stores and gas stations now carry CBD products.

The CBD beverage market, where solubility technology is very important, is expected to grow from $12M in the US in 2018 to over $1.6B in 2022, with a compound annual growth rate of 242% according the Brightfield Group. Internationally, the global cannabis-based beverages market is forecasted to reach $5.04B by 2026 according to Reports and Data. See, Cannabis-Based Beverages Market To Reach USD 5.04 Billion By 2026: Reports And Data. (2019, May 27). Retrieved Jun. 3, 2020, from https://www.globenewswire.com/news-release/2019/05/27/1850608/0/en/Cannabis-Based-Beverages-Market-To-Reach-US D-5-04-Billion-By-2026-Reports-And-Data.html.

Despite the rapidly growing consumer demand for CBD, key roadblocks such as lack of solubility, inconsistencies in dosing, and uncertain product shelf life are causing many established firms in the food, beverage, nutraceutical, and pharmaceutical industries to hesitate entering the CBD market.

Many companies are waiting for the necessary water-soluble technology to emerge that meets quality, aesthetic, and shelf-life standards for water-based products, such as topical creams and beverages. Existing so-called water-soluble CBD formulations are simply not good enough for widespread market adoption. Existing methods create milky solutions which lack aesthetic appeal for consumers. Furthermore, the CBD is unstable in the solution resulting in products with a short shelf life not viable for largescale distribution. While there are companies already offering CBD beverages, these firms are generally limited to serving regional geographies and many have come under fire for misrepresenting the actual CBD content of their beverages. For the cannabis beverage marketplace to scale up and mature to its potential, new dimensions of solubility technology are of utmost importance.

Full-spectrum organic hemp has over 400 unique botanical compounds (such as cannabinoids, flavonoids, terpenes and other plant matter), each of which differ in plant matter content, density, molecular structure, and solubility. This diversity of botanical matter in full-spectrum hemp represents the difficulty existing methods face in solubilizing hemp-derived CBD and other active ingredients. Generally, existing methods to solubilize CBD are only able to solubilize the isolated CBD molecule. Existing methods that claim to solubilize full-spectrum hemp, in fact, filter out much of the plant matter in their process and create inherently unstable solutions.

The water-soluble technology and delivery platform of the present invention represents a unique plant-based operating system that can solubilize a diverse array of lipophilic drug actives, other compounds, and plant matter, as is found in full-spectrum hemp. The present invention provides a platform that dynamically interfaces with the varied plant matter found in natural botanical extracts to create water-soluble solutions. The present invention provides superior product efficacy, minimizes adverse side effects, increases consumer appeal, and radically reduces production costs. Due to improved bio-availability and onset of action time, producers can therefore use 2 to 20 times less of the underlying plant material in their products to deliver the same or even superior experiences to consumers. The present invention solves the limitations of present water-soluble cannabis methods by creating full-spectrum solutions that are crystal clear, highly shelf stable, and easily used in a wide array of product applications.

While cannabis and hemp-derived CBD represent areas of immediate market demand, the present invention has applications far beyond the realm of cannabis. The present invention is capable of enhancing the beneficial impact of herbs and other botanicals that have been used for centuries in traditional medicine, such as turmeric, that are now in the midst of a modern renaissance and widespread adoption in modern lifestyles. According to Grandview Research, the global market for turmeric and curcumin, the active ingredient extracted from turmeric, is set to surpass $1.3B by 2025. See, Curcumin Market Size, Share: Industry Report, 2020-2027. (2020, April). Retrieved Jun. 17, 2020, from https://www.grandviewresearch.com/industry-analysis/turmeric-extract-curcumin-market. The present invention can play a key role in increasing the bio-availability of a material, such as turmeric, which is poorly water-soluble, and opens up new dimensions of product applications.

The present invention also has key potential applications for entheogenic plants which have been used for hundreds of years in various native traditions and are now also experiencing a renaissance in medical research. An entheogen is a psychoactive substance that induces alterations in perception, mood, consciousness, cognition, or behavior for the purposes of engendering physiological and psychological healing or spiritual development. These include, but are not limited to, ayahuasca, san pedro, peyote, (echinopsis pachanoi), iboga (tabernanthe iboga), and psilocybin mushrooms. Psilocybin research in particular is gaining increasing attention in research from leading universities including NYU and John Hopkins. See, Ross, S., Bossis, A., Guss, J., Agin-Liebes, G., Malone, T., Cohen, B., . . . Schmidt, B. L. (2016). Rapid and sustained symptom reduction following psilocybin treatment for anxiety and depression in patients with life-threatening cancer: A randomized controlled trial. Journal of Psychopharmacology, 30(12), 1165-1180. doi:10.1177/0269881116675512. Research indicates that entheogenic plants could have the potential to treat depression, anxiety, post-traumatic stress disorder, addiction and other mental health conditions. The present invention has the potential to enhance the bio-availability of medicines such as these and also offer greater consistency and control in dosing.

The present invention also has important potential applications for psychedelic and synthetic psychedelics such as LSD (Lysergic acid diethylamide) and 3,4-methylenedioxymethamphetamine, also known as MDMA, which has been granted Breakthrough Therapy status by the FDA for the treatment of posttraumatic stress disorder. See, Carpenter, D. (2020, May 12). Psychedelic Pioneer Rick Doblin On FDA Trials Of MDMA: Most Important Reality Check Of MAPS' 34-Year History. Retrieved Jun. 17, 2020, from https://www.forbes.com/sites/davidcarpenter/2020/05/12/psychedelic-pioneer-rick-doblin-on-fda-trials-of-mdma-most-important-reality-check-of-maps-34-year-history/. In some embodiments, water-soluble psychedelic active ingredients such as psilocybin may also be incorporated. Each active ingredient may be regulated differently in various markets and in more restricted markets, production, marketing, and usage could be limited by US or foreign federal, state, provincial, or local law. The compositions and methods including any regulated active should be appropriately practiced in consideration with such laws.

Dietary supplements and nootropics are also market categories that can benefit greatly from the present invention, as most products face problems of low-bioavailability and poor solubility.

In addition, the pharmaceutical industry, which struggles with poor solubility in the vast majority of new drugs, can also benefit immensely from the present invention.

In summary, the present invention provides a unique platform that bridges the gap between the potential of plant-based medicines to transform people's lives, and the actualization of that potential. The present invention is a master key that unlocks the full potential of plants and supplements by improving bio-availability, stability, and dosing precision of these plants. The present invention enables ancient plants, such as cannabis, to be offered in modern products for the modern consumer marketplace, while also retaining its original organic power. The present invention also provides a platform that can successfully incorporate water-insoluble, poorly water-soluble, or poorly permeable isolated molecules, as well as full plant botanical extracts or powders completely and seamlessly.

### Cannabis and Cannabinoids

The cannabis plant is sub-classified as Cannabis Sativa L., Indica, and Rudelaris. The primary distinguishing factor between each subtype is the varying levels of the cannabinoids Delta-9 Tetrahydrocannabinol (i9-THC) and Cannabidiol (CBD). Cannabis sativa L. sub-species, also known as hemp, is a variety that produces high levels of CBD and low levels of 9-THC. Cannabis indica, also known as marijuana, is a variety that generally produces a high levels of Δ9-THC and very low levels of CBD. In addition to Δ9-THC and CBD, cannabis is rich in over 100 other cannabinoids as well as terpenes and flavonoids several of which have been shown to have health benefits individually. Cannabinoids produced by the cannabis plant are known as phyto-cannabinoids which mimic the endocannabinoids produced naturally in the human body. Specifically, phyto-cannabinoids produce a therapeutic effect via endocannabinoid system in the human body. The cannabinoid receptors of the human endocannabinoid system have two main sub-types, CB1 and CB2, which are distributed throughout the central nervous system and in many peripheral tissues including the immune system, reproductive system, gastrointestinal tract, sympathetic ganglia, endocrine glands, arteries, lungs, and heart. Properties of CB receptor agonists have gained great clinical interest for their therapeutic effects. CB receptor agonists are effective pain relievers, muscle relaxants, immunosuppressants, anti-inflammatory agents. These receptors also have powerful anti-allergic, mood improvement, appetite improvement, anti-emesis, intra-ocular pressure reduction, bronchodilation, and anti-neoplastic effects. CB receptor antagonists have been studied as treatments for obesity, addictions or substance dependency, schizophrenia, Parkinson's disease, and Alzheimer's disease.

There are many challenges associated with water-insoluble (hydrophobic) materials. One of the main limitations with the delivery of cannabis extracts is the hydrophobic nature of cannabinoids which renders them water-insoluble and, therefore, inefficiently absorbed in humans. This hydrophobic property leads to significant limitations including: (1) Inconsistent dosing of cannabinoids in products; (2) Inconsistent onset of action once ingested in the human system; and (3) Low bioavailability in the body.

The present invention applies to incorporate the poorly water-soluble actives in lipid carriers and highly water-soluble actives in aqueous carriers thereby simultaneously delivering a wide variety of actives in a single composition. The poorly water-soluble molecules that can be incorporated in the lipid phase includes but is not limited to isolated molecules such as CBD, CBG, CBN, THC, melatonin, vitamin D, flavors, dyes and fragrances, and so on. The highly water-soluble molecules that can be incorporated in the same composition within the aqueous phase includes but is not limited to isolated molecules such as xylitol, sugars, dyes, flavors, and fragrances, and so on. Poorly water-soluble plant, algae, fungi, or animal-based extracts that can also be incorporated in the lipid phase includes but is not limited to full-spectrum hemp extract, turmeric powder, sea-weed extract, fish oil, and so on. The highly water-soluble plant, algae, fungi, or animal-based extracts that can be incorporated in the same composition within the aqueous phase includes but is not limited to aloe vera extract, green tea extract, and other such extracts.

In the present invention the active agent can be selected from full-spectrum cannabis extracts, or one or more of the following cannabinoids: cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), delta-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), delta-7-cis-iso-tetrahydrocannabivarin, delta-8-tetrahydrocannabinolic acid (.DELTA⁸-THCA), delta-8-tetrahydrocannabinol (DELTA⁸-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC).

The compositions of the present invention comprise from about 0.0001% to 99.9% by weight of active ingredients. In further embodiments, the compositions of the present invention comprise from about 0.1% to about 99% by weight, from about 0.25% to about 95% by weight, from about 0.5% to about 90% by weight, from about 1% to about 80% by weight, from about 5% to about 75% by weight, and from about 10% to about 50% by weight of the active ingredient. In some embodiments, the concentrate may comprise about 0.0001%, 0.001%, 0.01%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 2%, 2.5%, 3%, 3.5%, 4%, 4.5%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95% of active ingredient by weight.

Other Active Ingredients

Proteins and Polypeptides

In other embodiments the active ingredient can be a protein or polypeptide. The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof. It is recognized that in some embodiments the protein or polypeptide can be water-soluble. In further embodiments the protein or polypeptide can be formulated into a liposome, and this liposome complex can be considered as the active ingredient which is then further incorporated into the compositions of the present invention.

Water-Insoluble or Poorly Water Soluble Active Ingredients

In various embodiments the water insoluble or poorly water soluble active ingredient is selected from the group consisting of essential oils (i.e. also known as plant extracts or botanical extracts), pharmaceutical drug actives, vitamins, fish oil, milk derivatives, fragrances, flavorings, colorings, sweeteners, taste-enhancers, anti-oxidants, and mixtures thereof.

In some embodiments, the water-insoluble or poorly water soluble active ingredient may be selected from the group consisting of cannabis extract, hemp oil, human breast milk, cannabinoids, natural phytocannabinoids, organic cannabinoids, endocannabinoids, cannabinoid analogs, cannabinoid derivatives, synthetic cannabinoids, cannabinoid receptor agonists, and mixtures thereof. In some embodiments, the water-insoluble or poorly water soluble active ingredient may be selected from the group consisting of LSD, MDMA, and any other psychedelics.

Water-Soluble Active Ingredients

In other embodiments, the compositions can further comprise one or more water-soluble active ingredients, which are readily formulated into the compositions. The water-soluble active ingredient can be selected from the group consisting of water-soluble plant extracts, pharmaceutical drug actives, vitamins, fragrances, flavorings, colorings, sweeteners, taste-enhancers, anti-oxidants, and mixtures thereof. The water-soluble active can be selected from the group consisting of aloe vera extract, green tea extract, stevia leaf extract, and mixtures thereof. In some embodiments, the water-soluble active ingredients may further comprise a psychedelic, such as psilocybin.

### Components of the Compositions

Lipophilic Components

The compositions of the present invention comprise a lipophilic component. In some embodiments, the lipophilic component can be selected from the group consisting of oils, glycerides, waxes, alcohols, hydroalcoholic mixtures, monoglycerides, diglycerides, triglycerides, cannabis oil, cannabis-seed oil, coconut oil, cottonseed oil, borage oil, soybean oil, safflower oil, sunflower oil, castor oil, corn oil, olive oil, palm oil, peanut oil, almond oil, sesame oil, rapeseed oil, peppermint oil, poppy seed oil, canola oil, including whole and fractionated oil forms of any of the foregoing, and mixtures thereof.

The lipophilic component in some embodiments has an HLB value of from about zero to about 7, or from about 0 to about 4.

The lipid component can comprise from about 0.0001% to 90% by weight of active ingredients and the aqueous carrier can comprise from 0% to less than 90% by weight of active ingredients. The lipid carriers can be oils or oil extracts such as sunflower oil, hemp seed oil, peanut oil, olive oil, fractionated coconut oil, processed oils and so on.

The coconut oil, palm oil, kernel oil, peanut oil, and sesame oil is known to have major components having a medium carbon chain length between C8-C12 and are primarily the mixtures of monoglycerides, diglycerides and triglycerides of the fatty acid esters.

The olive oil, soybean oil, safflower oil, and such have major components having a long chain length between C14-C22 and are primarily mixtures of monoglycerides, diglycerides and triglycerides of the fatty acid esters. The fractionated forms of these oils are separated components of these glycerides of fatty acid esters. The lipid carriers are also known to have an impact on the enhanced permeability of the actives through the epithelial or mucosal membrane. Other components in the lipid carriers that have emulsifying and surface-active properties are added in addition to the oils, which have an HLB values between 12-15. The emulsifying carriers can be comprised of sorbitan esters, ethoxylated sorbitan esters, polyalcohols, ethoxylated alky phenols, amine derivatives, amide derivatives, alkylpolyglucosides, ethyleneoxide-propylene-oxide copolymers, thiols or derivatives thereof, poloxamers, pegylated fatty acid esters, polysorbates, sugar ester, lecithin, bile salts, albumin, alcohols, and mixtures thereof. The resultant HLB values of the carrier and emulsifying lipids range between 7-10 to lower the surface tension of active ingredients thereby producing nanoparticulate or nanoencapsulated systems of active ingredients.

The lipophilic component of the preconcentrate may comprise one or more different components and the term “lipophilic component” used in the singular is intended to include one or more lipophilic components. The lipophilic component may include plant-based oils, glycerides, waxes, alcohols, hydroalcoholic mixtures, whole and fractionated oil forms of any of the foregoing, and mixtures thereof, including whole or fractionated oil forms. In some embodiments, the lipophilic component may be a glyceride, such as a monoglyceride, a diglyceride, and/or a triglyceride and mixtures thereof.

In some embodiments, the lipophilic component may be one or more of a monoglyceride, diglyceride, and/or triglyceride of C6 to C30 carboxylic acids, and mixtures thereof, wherein the C6 to C30 carboxylic acids are selected from fully saturated carboxylic acids, and/or carboxylic acids optionally having 1, 2, or 3 unsaturated carbon-carbon bonds which can be variously positioned along the carbon skeleton of the carboxylic acid. In some embodiments, if the C6 to C30 carboxylic acid comprises one or more unsaturated carbon-carbon bonds, each may independently be in either the cis or trans configuration.

In some embodiments, the lipophilic component may be referred to by Chemical Abstracts Service (CAS) registry number, i.e. the “CAS number”. A CAS number may define a substance, class of substances, or individual molecular structure. Stereochemistry and/or configurations may be defined by a CAS number. To a person of skill in the art, a CAS number is a well-recognized, defining, and specific description of a substance or molecule. In some embodiments, exemplary and non-limiting lipophilic components may correspond to one or more of the following CAS registry numbers CAS 92045-31-3, CAS 91744-32-0, CAS 85536-07-8, CAS 91052-28-7, CAS 91744-09-1, CAS 91744-13-7, CAS 24529-88-2; CAS 85251-77-0, CAS 85536-06-7, CAS 91744-20-6, and CAS 122-32-7, and mixtures thereof. In some embodiments, the lipophilic component may be a glyceride such as cocoglycerides; glyceryl monocaprylocaprate (C8-10 mono and diglycerides); glycerides, C14-18 and C16-18-unsatd. mono-, di- and tri-; glycerides, C16-18 and C18-unsatd. mono-; glycerides, C14-18 and C16-22-unsatd. mono- and di-; (1-hexadecanoyloxy-3-hydroxypropan-2-yl) octadecenoate; glycerides, C8-18; glycerides, C16-18 and C18-unsatd. mono-, di and tri-; 1,2,3-tri(cis-9-octadecenoyl)glycerol; and combinations thereof.

In some embodiments, the lipophilic component comprises from about 0.1% to about 99.9%, or from about 1% to about 80%, or from about 5% to about 50%, or about 10% to about 40%, or from about 10% to about 25% by weight of the pre-concentrate. In some embodiments, each lipophilic component comprises about 1, 2, 3, 4, 5, 10, 15, 20, 25, 30, 35, 40, 45, 50, 55, 60, 65, 70, 75, 80, 85, 90, or 95% of the pre-concentrate by weight.

Surface Active Agents

The compositions of the present invention comprise a surface-active agent. In some embodiments, the surface-active agent can be selected from the group consisting of sorbitan esters, ethoxylated sorbitan esters, polyalcohols, ethoxylated alky phenols, amine derivatives, amide derivatives, alkylpolyglucosides, ethyleneoxide-propylene-oxide copolymers, thiols or derivatives thereof, poloxamers, pegylated fatty acid esters, polysorbates, sugar ester, lecithin, bile salts, albumin, alcohols, and mixtures thereof.

The lipid component can comprise from about 0.0001% to 90% by weight of active ingredients and the aqueous carrier can comprise from 0% to less than 90% by weight of active ingredients. The lipid carriers can be oils or oil extracts such as sunflower oil, hemp seed oil, peanut oil, olive oil, fractionated coconut oil, processed oils and so on.

In some embodiments, the surface active agent may comprise a hydrophilic head group and one or more C10-C30 fatty acid chains. The hydrophilic head group may be any suitable group, such as a sugar ester, aliphatic alcohols, aliphatic polyhydric alcohols, saccharides, disaccharides, aliphatic amines, aliphatic polyamines, aliphatic amino alcohols, aliphatic amino polyhydric alcohols, aliphatic polyamino alcohols, aliphatic polyamino polyhydric alcohols, and combinations thereof. In some embodiments, hydrophilic head group is ethylene glycol, propylene glycol, 1,3-propane diol, 1,2-butane diol, 1,3-butane diol, 1,4-butane diol, glycerol, glyceraldehyde, 1-hydroxy-2-amino ethane, or combinations thereof. In some embodiments, the hydrophilic head group is glycerol. In some embodiments, the hydrophilic head group is a sugar ester such as a fructose ester, sucrose ester, or glucose ester. In some embodiments, the hydrophilic head group is a sucrose ester.

In some embodiments, the surface active agent is a monoglyceride, a diglyceride, a triglyceride, and/or combinations thereof. In some embodiments, the surface active agent comprises a mono-, di-, or triglyceride of a C10-C30 fatty acid. The C10-C30 fatty acid may be saturated or unsaturated and may contain one or more —OH substituents. In some embodiments, each C10-C30 fatty acid is independently modified with one or more ethylene glycol groups, one or more propylene glycol groups, or a mixture of one or more ethylene glycol and propylene glycol groups. Each C10-C30 fatty acid, including any substitutions thereto, in a di- or triglyceride may be the same or different. In some embodiments, a hydrophilic head group is covalently bonded to one or more C10-C30 fatty acids directly via an ester linkage to the C10-C30 fatty acid, and at least one of the hydroxy substituents of at least one of the C10-C30 fatty acid chains may optionally be ethoxylated, propoxylated, or mixed ethoxylated/propoxylated. In some embodiments, a hydrophilic head group is covalently bonded to one or more C10-C30 fatty acids indirectly via an ester or ether, linkage to an optional intervening ethoxy, propopxy, or mixed ethoxy/propoxy group. In some embodiments, the hydrophilic head group is covalently bonded to one or more C10-C30 fatty acids indirectly via an ester or ether linkage to an intervening ethoxy, propopxy, or mixed ethoxy/propoxy group, and at least one of the hydroxy substituents of at least one of the C10-C30 fatty acid chains may be optionally ethoxylated, propoxylated, or mixed ethoxylated/propoxylated.

In some embodiments, the surface active agent is an ester of a sugar. In some embodiments, surface active agent may be an ester of sucrose, fructose, or glucose. In some embodiments, the surface active agent may be an ester of sucrose or fructose. In some embodiments, the surface active agent may be an ester of sucrose. In some embodiments, the surface active agent is an ester of sucrose having 1, 2, 3, 4, 5, 6, 7, or 8 esterified positions. In some embodiments, the surface active agent is an ester of sucrose having 1, 2, or 3 esterified positions. In some embodiments, the esterified positions may be adjacent or non-adjacent. In some embodiments, all esterified positions are on the glucose ring. In some embodiments, all esterified positions are on the fructose ring. In some embodiments, the surface active agent is an ester of sucrose having 1 esterified position.

In some embodiments, the sucrose head group may be esterified at any number of available —OH bearing positions with a C10-C30 fatty acid. The C10-C30 fatty acid may be saturated or unsaturated and may contain one or more —OH substituents. In some embodiments, each C10-C30 fatty acid is independently modified with one or more ethylene glycol groups, one or more propylene glycol groups, or a mixture of one or more ethylene glycol and propylene glycol groups. Each C10-C30 fatty acid, including any substitutions thereto, in a di- or triglyceride may be the same or different. In some embodiments, a hydrophilic head group is covalently bonded to one or more C10-C30 fatty acids directly via an ester linkage to the C10-C30 fatty acid, and at least one of the hydroxy substituents of at least one of the C10-C30 fatty acid chains may optionally be ethoxylated, propoxylated, or mixed ethoxylated/propoxylated. In some embodiments, a hydrophilic head group is covalently bonded to one or more C10-C30 fatty acids indirectly via an ester or ether, linkage to an optional intervening ethoxy, propopxy, or mixed ethoxy/propoxy group. In some embodiments, the hydrophilic head group is covalently bonded to one or more C10-C30 fatty acids indirectly via an ester or ether linkage to an intervening ethoxy, propopxy, or mixed ethoxy/propoxy group, and at least one of the hydroxy substituents of at least one of the C10-C30 fatty acid chains may be optionally ethoxylated, propoxylated, or mixed ethoxylated/propoxylated. In some embodiments, the sucrose head group may alternatively be any —OH bearing sugar molecule, including glucose, sucrose, lactose, galactose, maltose, trehalose, xylose, isomaltose, mannose, tagatose, or trehalulose, including stereoisomers.

In some embodiments, the surface active agent may have a CAS number corresponding to CAS 61788-85-0, CAS 188734-82-9, CAS 57176-33-7, CAS 70142-34-6, CAS 68953-20-8, CAS 31835-02-6, CAS 13039-40-2, CAS 61791-12-6, CAS 854374-08-6, CAS 122636-35-5, CAS 122636-36-6, CAS 9005-64-5, CAS 90005-65-6, CAS 145-42-6, CAS 388610-12-6, and mixtures thereof.

In some embodiments, the surface active agent is one or more of sorbitan esters, ethoxylated sorbitan esters, polyalcohols, ethoxylated alky phenols, amine derivatives, amide derivatives, alkylpolyglucosides, ethyleneoxide-propylene-oxide copolymers, thiols or derivatives thereof, poloxamers, pegylated (ethoxylated) fatty acid esters, propoxylated fatty acid esters, mixed ethoxylated/propoxylated fatty acid esters, pegylated (ethoxylated) fatty acid triglycerides, propoxylated fatty acid triglycerides, mixed ethoxylated/propoxylated fatty acid triglycerides, pegylated (ethoxylated) hydroxy substituted fatty acid triglycerides, propoxylated hydroxy substituted fatty acid triglycerides, mixed ethoxylated/propoxylated hydroxy substituted fatty acid triglycerides, wherein said fatty acids are optionally unsaturated, polysorbates, sugar ester, lecithin, bile salts, albumin, alcohols, and mixtures thereof.

In some embodiments, the surface active agent is one or more of ethoxylated castor oil (polyoxyethylene castor oil); RO 40; BY 140; PEG Castor oil; PEG-10 Castor oil, PEG-100 Castor oil, PEG-1 Castor oil, PEG-15 Castor oil, PEG-2 Castor oil, PEG-20 Castor oil, PEG-200 Castor oil, PEG-25 Castor oil, PEG-26 Castor oil, PEG-3 Castor oil, PEG-30 Castor oil, PEG-33 Castor oil, PEG-35 Castor oil, PEG-36 Castor oil, PEG-4 Castor oil, PEG-40 Castor oil, PEG-5 Castor oil, PEG-50 Castor oil, PEG-54 Castor oil, PEG-55 Castor oil, PEG-60 Castor oil, PEG-8 Castor oil, PEG-9 Castor oil, polyethoxylated castor oil, polyethylene glycol (100) castor oil, polyethylene glycol (11) castor oil, polyethylene glycol (15) castor oil, polyethylene glycol (25) castor oil, polyethylene glycol (26) castor oil, polyethylene glycol (3) castor oil, Polyethylene glycol (30) castor oil, Polyethylene glycol (33) castor oil, Polyethylene glycol (35) castor oil, Polyethylene glycol (5) castor oil, Polyethylene glycol (50) castor oil, Polyethylene glycol (54) castor oil, Polyethylene glycol (55) castor oil, Polyethylene glycol (60) castor oil, Polyethylene glycol 1000 castor oil, Polyethylene glycol 1800 castor oil, Polyethylene glycol 200 castor oil, Polyethylene glycol 2000 castor oil, Polyethylene glycol 400 castor oil, Polyethylene glycol 450 castor oil, Polyethylene glycol 500 castor oil, Polyoxyethylene (10) castor oil, Polyoxyethylene (100) castor oil, Polyoxyethylene (11) castor oil, Polyoxyethylene (15) castor oil, Polyoxyethylene (2) castor oil, Polyoxyethylene (20) castor oil, Polyoxyethylene (200) castor oil, Polyoxyethylene (25) castor oil, Polyoxyethylene (26) castor oil, Polyoxyethylene (3) castor oil, Polyoxyethylene (30) castor oil, Polyoxyethylene (33) castor oil, Polyoxyethylene (35) castor oil, Polyoxyethylene (36) castor oil, Polyoxyethylene (4) castor oil, Polyoxyethylene (40) castor oil, Polyoxyethylene (5) castor oil, Polyoxyethylene (50) castor oil, Polyoxyethylene (54) castor oil, Polyoxyethylene (55) castor oil, polyoxyethylene (60) castor oil, polyoxyethylene (8) castor oil, polyoxyethylene (9) castor oil, and combinations thereof.

In some embodiments, the surface active agent is one or more of Sucrose stearate, sucrose palmitate, sucrose laurate, sucrose behenate, sucrose oleate, sucrose erucate, sucrose ester of mixed fatty acids, fructose stearate, fructose palmitate, fructose laurate, fructose behenate, fructose oleate, fructose erucate, fructose ester of mixed fatty acids, glucose stearate, glucose palmitate, glucose laurate, glucose behenate, glucose oleate, glucose erucate, glucose ester of mixed fatty acids, lactose stearate, lactose palmitate, lactose laurate, lactose behenate, lactose oleate, lactose erucate, lactose ester of mixed fatty acids, including saturated and —OH modified fatty acids and, optionally, ethoxy, propoxy, and/or mixed ethoxy/propoxy groups covalently bonded to the sugar head group, to the —OH groups on the fatty acid chains, and/or intervening the sugar ester to fatty acid ester bond.

In some embodiments, the surface active agent may have a general structure according to Formula I. In some embodiments, each occurrence of m may be independently selected from the group consisting of 6, 7, 8, 9, 10, 11, 12, 13, 14, 15, 16, 17, 18, 19, 20, 21, 22, 23, 24, 25, 26, 27, 29, 29, and 30. In some embodiments, each occurrence of n may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, each occurrence of n may include a propoxy group in place of an ethoxy group. While the triglyceride is shown, the mono- and di-glycerides are contemplated.

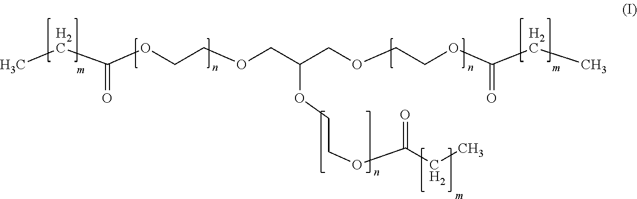

In some embodiments, the surface active agent may have a general structure according to Formula II. In some embodiments, each occurrence of m may be independently selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 29, 29, and 30, such that the total value of m in a single fatty acid chain is no greater than 29. In some embodiments, each occurrence of n and/or p may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, each occurrence of n may include a propoxy group in place of an ethoxy group. While the triglyceride is shown, the mono- and di-glycerides are contemplated.

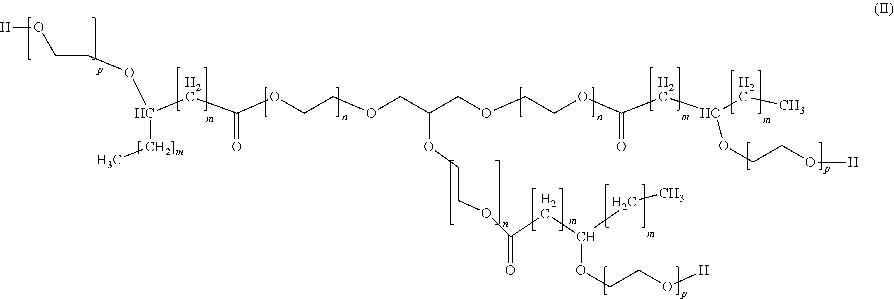

In some embodiments, the surface active agent may have a general structure according to Formula III. In some embodiments, each occurrence of m may be independently selected from the group consisting of 6, 7, 8, 9, 10, 11, 12, 13, 14, 15, 16, 17, 18, 19, 20, 21, 22, 23, 24, 25, 26, 27, 29, 29, and 30. In some embodiments, each occurrence of n may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, each occurrence of n may include an ethoxy group in place of a propoxy group. While the triglyceride is shown, the mono- and di-glycerides are contemplated.

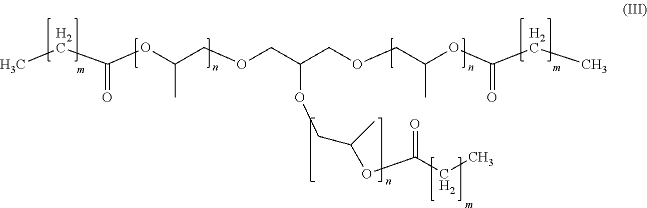

In some embodiments, the surface active agent may have a general structure according to Formula IV. In some embodiments, each occurrence of m may be independently selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 29, 29, and 30, such that the total value of m in a single fatty acid chain is no greater than 29. In some embodiments, each occurrence of n and/or p may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, each occurrence of n may include an ethoxy group in place of a propoxy group. While the triglyceride is shown, the mono- and di-glycerides are contemplated.

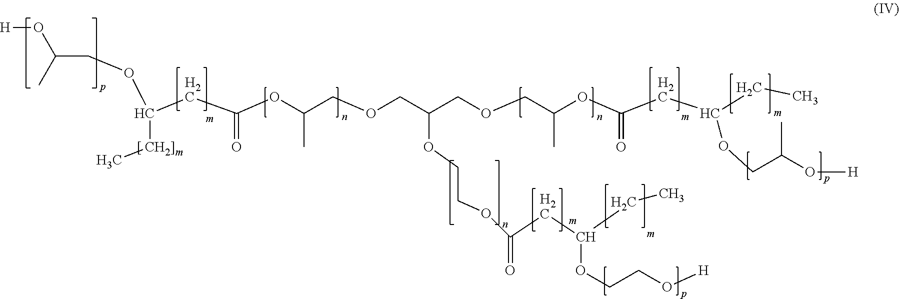

In some embodiments, the surface active agent may have a general structure according to Formula V. In some embodiments, each occurrence of m may be independently selected from the group consisting of 6, 7, 8, 9, 10, 11, 12, 13, 14, 15, 16, 17, 18, 19, 20, 21, 22, 23, 24, 25, 26, 27, 29, 29, and 30. In some embodiments, each occurrence of n may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, any of the —OH groups of the sucrose molecule may or may not be modified with the ester and/or ether shown. In some embodiments, each occurrence of n may include a propoxy group in place of an ethoxy group.

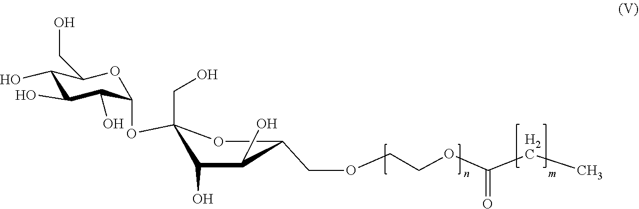

In some embodiments, the surface active agent may have a general structure according to Formula VI. In some embodiments, each occurrence of m may be independently selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 29, 29, and 30, such that the total value of m in a single fatty acid chain is no greater than 29. In some embodiments, each occurrence of n and/or p may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, any of the —OH groups of the sucrose molecule may or may not be modified with the ester and/or ether shown. In some embodiments, each occurrence of n may include a propoxy group in place of an ethoxy group.

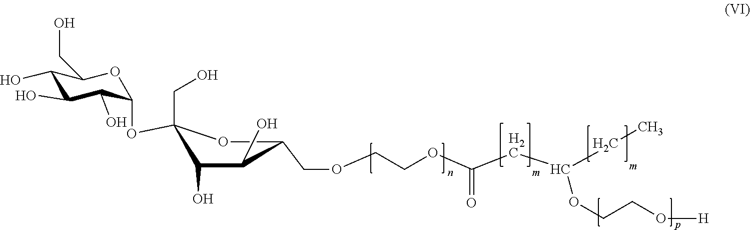

In some embodiments, the surface active agent may have a general structure according to Formula VII. In some embodiments, each occurrence of m may be independently selected from the group consisting of 6, 7, 8, 9, 10, 11, 12, 13, 14, 15, 16, 17, 18, 19, 20, 21, 22, 23, 24, 25, 26, 27, 29, 29, and 30. In some embodiments, each occurrence of n may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, any of the —OH groups of the sucrose molecule may or may not be modified with the ester and/or ether shown. In some embodiments, each occurrence of n may include an ethoxy group in place of a propoxy group.

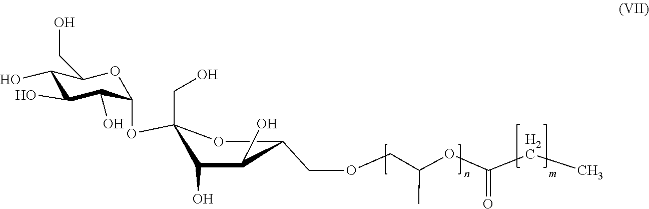

In some embodiments, the surface active agent may have a general structure according to Formula VIII. In some embodiments, each occurrence of m may be independently selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 29, 29, and 30, such that the total value of m in a single fatty acid chain is no greater than 29. In some embodiments, each occurrence of n and/or p may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. Each occurrence of m may omit a hydrogen atom in order to yield one or more C═C, or unsaturated, bonds, at any viable position. In some embodiments, any of the —OH groups of the sucrose molecule may or may not be modified with the ester and/or ether shown. In some embodiments, each occurrence of n may include an ethoxy group in place of a propoxy group.

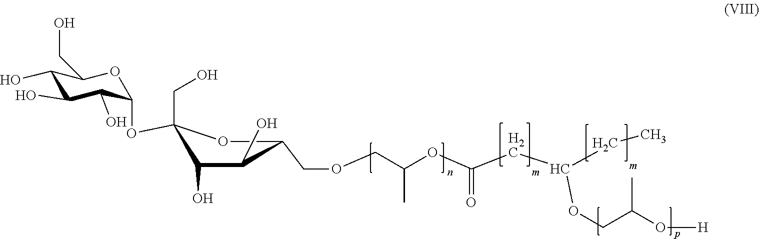

In some embodiments, the surface active agent may have a general polysorbate structure according to Formula IX. In some embodiments, m may be independently selected from the group consisting of 6, 7, 8, 9, 10, 11, 12, 13, 14, 15, 16, 17, 18, 19, 20, 21, 22, 23, 24, 25, 26, 27, 29, 29, and 30. In some embodiments, each occurrence of w, x, y, and z may be independently selected from the group consisting of less than about 1000, or less than about 900, or less than about 800, or less than about 700, or less than about 600, or less than about 500, or less than about 400, or less than about 300, or less than about 200, or less than about 100, or less than about 75, or less than about 70, or less than about 65, or less than about 60, or less than about 55, or less than about 50, or less than about 45, or less than about 40, or less than about 35, or less than about 30, or less than about 25, or less than about 20, or less than about 15, or less than about 10, or less than about 8, or less than about 6, or less than about 5, or less than about 4, or less than about 3, or less than about 2, or 1, or 0, or any ranges, inclusive thereof, or any values in-between the given values. In some embodiments, each occurrence of w, x, y, and z is selected from the group consisting of 1, 2, 3, 4, 5, 6, 7, 8, 9, 10, 11, 12, 13, 14, 15, 16, 17, 18, 19, and 20. In some embodiments, w+x+y+z is about 20. In some embodiments, each occurrence of m may omit a hydrogen atoms to yield one or more C═C, or unsaturated, bonds, at any viable position. In some embodiments, each occurrence of x, y, and/or z may include a propoxy group in place of an ethoxy group.

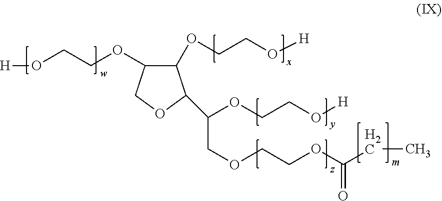

The surface-active agents in some embodiments have an HLB value of from about 10 to about 13, or from about 11 to about 12. In some embodiments, the surface-active agent comprises from about 0.1% to about 99.9%, or from about 1% to about 80%, or from about 5% to about 50%, or about 10% to about 40%, or from about 10% to about 25% by weight of the pre-concentrate. In some embodiments, each surface active agent comprises about 1, 2, 3, 4, 5, 10, 15, 20, 25, 30, 35, 40, 45, 50, 55, 60, 65, 70, 75, 80, 85, 90, or 95% of the pre-concentrate by weight.

Water or Aqueous Carriers

The aqueous carriers can be water, natural, or artificially flavored beverages, juices, tea, coffee, dairy or non-dairy milk and so on. The aqueous carriers can comprise mixtures of water and other miscible liquids such as ethanol, glycerol, and the like. The water-soluble components in a concentrate can be incorporated by mixing a concentrate into the aqueous carrier. Mixing of lipid carriers with poorly water-soluble actives and aqueous carriers with water-soluble actives can provide a delivery system to administer:
1) One or more poorly water-soluble actives
2) One or more water-soluble actives
3) A combination of both thereof.

Methods of Preparation

The lipophilic component can be used in their non-processed or processed form depending on the lipophilicity of the active ingredients. The processing of lipid carriers allows them to select the components of glycerides within the oils that are suitable carriers for the active ingredients. The combination of whole or fractionated oils are mixed in ratios that are calibrated to each active ingredient desired to be incorporated. In some embodiments, the HLB values of the lipophilic component(s) is adjusted between 0-7 to allow the high solubility of active ingredients into the lipid carriers. In some embodiments, the HLB values of the lipophilic component(s) is adjusted between 0-4. The surface-active agents are then mixed with lipophilic component(s) to lower the surface tension of the system consisting of poorly water-soluble active in the lipid carrier itself.

In some embodiments, the mixtures are prepared at room temperature by simple mixing and are not heated. In some embodiments, as appropriate the mixtures can be heated, or heated with mixing from about 40° C. to about 100° C.

In alternative embodiments, the lipid carriers are processed and optionally mixed using sonic energy to create an individual micelle by exposing the lipids to a range of frequencies between 180-990 Hz. These frequencies are produced in specific sequences of varying exposure times which enhance both the stability of the compositions and the efficiency with which it solubilizes actives later in the process. The total timing of frequency exposure can range from about 6 hours to 72 hours.

The resultant compositions of the present invention are referred to as the “Operating System” or, simply, “OS”. The active ingredient is then incorporated into the OS using a simple blending or mixing apparatus such as mixers or magnetic stirrers. The active ingredients can be heated to an elevated room temperature to improve the mixing and solubilization of these into OS. This OS now comprises one or more active ingredients such as full-spectrum hemp extract or the isolated CBD molecule is described as:
1). a water-soluble platform,
2). a highly permeable platform,
3). a highly bioavailable platform,
4). a platform for bypassing first pass liver metabolism, and
5). a combination thereof.

The poorly water-soluble active ingredients are mixed in the OS and, if needed, the highly water-soluble active ingredients are mixed into the aqueous carrier separately. Upon mixing of the actives completely in each of the said systems, the lipid and aqueous phase can be mixed to form a final product composition that is either in a water-in-oil-emulsion; or liquid crystalline phase (gel-like phase); or as an oil-in-water-microemulsion. The final composition can be a finished product in itself or further used as a raw material in the intended finished product. The mixing of the lipid and aqueous phases is done using the simple blending or mixing equipment such as stirring, mechanical blender, or magnetic mixer.

### EXAMPLES

The following examples further described and demonstrate embodiments within the scope of the present invention. The Examples are given solely for purpose of illustration and are not to be construed as limitations of the present invention, as many variations thereof are possible without departing from the spirit and scope of the invention.

### Example 1: Preconcentrate Formulation

The following formulation is a preconcentrate composition containing a mixture of lipid carriers and surface-active agents. The lipid carriers and surface-active agents collectively form the OS as described earlier and are optionally processed through the range of sonic frequencies to form a structural conformity to incorporate the active ingredient that is poorly water-soluble.

The lipid carrier can be a whole or fractionated form of peanut oil, coconut oil, castor oil, olive oil, hemp seed oil, sesame oil, fish oil, sunflower oil, essential oils, and so on depending on the miscibility of the active ingredients. For example, these include a fractionated form of medium chain glycerides of capric acid, caprylic acid and lauric acid extracted from coconut oil, palm oil, cannabis seed oil and sesame oil in varying ratios. The other component is a surface-active base having a lipophilic head and a hydrophilic tail to reduce the surface tension and balance the HLB values of lipid carrier and active ingredients intended for forming a nano encapsulated systems that solubilize in aqueous medium. Table 1 provides a summary of these compositions, including the ranges of quantities of each component.

TABLE 1
Summary of the components and their quantities (% w/w) of
Example Preconcentrate Formulation

| Component | % w/w | | |
|---|---|---|---|
| Lipophilic component | 0.1-99.9% | w/w | Having an HLB value |
| (fractionated coconut oil, | | | of less than 0-7 |
| palm oil cannabis seed oil | | | |
| and sesame oil) | | | |
| Surface active agent | 0.1-99.9% | w/w | Having am HLB value |
| | | | of between 10-13 |
| Total | 100% | w/w | Aggregate value of |

HLB value between
7-10

This preconcentrate can be used to prepare a concentrate water a water-insoluble or poorly water-soluble active ingredient.

### Example 2: Concentrate Formulation A

Example 2 is based on a preconcentrate of Example 1 to make Formulation A which is a concentrate composition containing a mixture of lipid carriers and surface-active agents, and the active ingredients. The lipid carriers and surface-active agents collectively form the OS as described earlier and are processed through the range of sonic frequencies to form a structural conformity to incorporate the active ingredient that is poorly water-soluble. The active ingredient is heated at temperatures between 0° C. to 100° C. dependent on the maximum heat tolerated by the OS as well as active ingredient without decomposing due to heat. Upon heating, the active ingredient is solubilized into the OS to form a clear solution. This concentrate of water-soluble composition containing OS with active can be directly administered in capsules or as freeze-dried powders to be mixed with aqueous liquids within the gastrointestinal tract or to be mixed with a water-based aqueous carrier to form a gel, cream or clear solution. The lipids carrier can be a whole or fractionated form of peanut oil, coconut oil, castor oil, olive oil, hemp seed oil, sesame oil, fish oil, sunflower oil, essential oils, and so on depending on the miscibility of the active ingredients. For example, these include a fractionated form of medium chain glycerides of capric acid, caprylic acid and lauric acid extracted from coconut oil, palm oil, cannabis seed oil and sesame oil in varying ratios. The other component is a surface-active base having a lipophilic head and a hydrophilic tail to reduce the surface tension and balance the HLB values of the lipid carrier and active ingredients intended for forming a nano encapsulated system that solubilizes in aqueous medium. Table 2 provides a summary of these compositions, including the ranges of quantities of each component.

TABLE 2
Summary of the components and their quantities (% w/w) of
Example Concentrate Formulation A

| Component | % w/w | | |
|---|---|---|---|
| Hemp extract (or | 0.0001-90% | w/w | Having an HLB |
| equivalent active | | | value of 0-7 |
| ingredients) | | | |
| Lipophilic component | 0.1-99.9% | w/w | Having an HLB value |
| (fractionated coconut oil, | | | of less than 0-7 |
| palm oil cannabis seed oil | | | |
| and sesame oil) | | | |
| Surface active agent | 0.1-99.9% | w/w | Having an HLB value |
| | | | of between 10-13 |
| Total | 100% | w/w | Aggregate value of |

HLB value between
7-10

This concentrate can be incorporated into (1) the capsules or administered as freeze dried powder, tablets; or mixed with aqueous carriers to form (2) oral liquids, beverages, beverage additive, food additives, sub-lingual liquids, buccal liquids, intranasal liquids, mist or spray for oral or intranasal use; or mixed with (3) excipients for topical or transdermal application; or mixed with (4) excipients for mucosal applications such as oro-buccal patches, films, tablets, suppositories, etc.

Drug dissolution studies have been a long-accepted measure of drug active solubilization into simulated aqueous gastrointestinal fluids. The importance of the dissolution rate on clinical performance of drugs and drug delivery systems has long been recognized, as well as the property of dosage forms that contribute to the rate and extent of drug availability into the body. Dissolution analysis of pharmaceutical solid dosage forms has emerged as the single most important test that ensures the bioavailability and the quality of the product. See, Banakar, U. V. (1992). Pharmaceutical dissolution testing. New York: Marcel Dekker, pp. iv-vii.

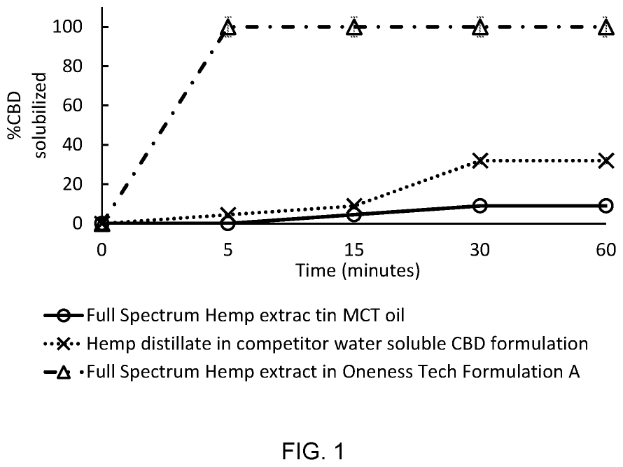

The graph in FIG. 1 shows the solubilization of model active, cannabidiol, dissolved in the simulated gastrointestinal aqueous fluid. The graph compares (1) Full-spectrum hemp extracted cannabidiol 100 mg in an aqueous Formulation A of the present invention versus (2) a commercially available distillated hemp extract cannabidiol 100 mg in aqueous formulation, which is marketed aqueous formulation of hemp; and (3) a full-spectrum hemp extracted cannabidiol 100 mg formulated in medium-chain triglyceride (MCT) oil, which is the present industry standard oil based formulation of hemp.

These results show that increased water solubilization is reported in as compared to that using only current aqueous formulations or standard oil-based formulations based on medium-chain triglycerides.

### Example 3: Concentrate Formulation B

Analogous preconcentrates for Formulation B can be prepared according to Example 1. Formulation B is a concentrate composition containing a mixture of lipid carriers and surface-active agents, and the active ingredients. The lipid carriers and surface-active agents collectively form the OS as described earlier are processed through the range of sonic frequencies to form a structural conformity to incorporate the active ingredient that is poorly water-soluble. The active ingredient is heated at temperatures between 0° C. to 100° C. dependent on the maximum heat tolerated by OS as well as the active ingredient without decomposing due to heat. Upon heating, the active ingredient is solubilized into the OS to form a clear solution. This concentrate of water-soluble composition containing OS with active can be directly administered as capsules or as freeze-dried powder to be mixed with aqueous liquids within the gastrointestinal tract or be mixed with water based aqueous carrier to form a gel, cream or clear solution.

The lipids carrier can be a whole or fractionated form of peanut oil, coconut oil, castor oil, olive oil, hemp seed oil, sesame oil, fish oil, sunflower oil, essential oils, and so on depending on the miscibility of the active ingredients. For example, these include a fractionated form of long chain glycerides of myristic acid, oleic acid, stearic acid, palmitic acid and arachidic acid extracted from olive oil, soybean oil, peanut oil, canola oil and macadamia oil in varying ratios. The other component is a surface-active base having a lipophilic head and a hydrophilic tail to reduce the surface tension and balance the HLB values of lipid carrier and active ingredients intended for forming a nano encapsulated system that solubilize in aqueous medium. Table 3 provides a summary of these compositions, including the ranges of quantities of each component.

TABLE 3
Summary of the components and their quantities (% w/w) of
Example Concentrate Formulation B

| Component | % w/w | | |
|---|---|---|---|
| Hemp extract (or | 0.0001-90% | w/w | Having an HLB value |
| equivalent active | | | of 0-7 |
| ingredients) | | | |
| Lipophilic component | 0.1-99.9% | w/w | Having an HLB value |
| (fractionated olive oil, | | | of less than 0-7 |
| soybean oil, peanut oil, | | | |
| canola oil and | | | |
| macadamia oil) | | | |
| Surface active agent | 0.1-99.9% | w/w | Having am HLB value |
| | | | of between 10-13 |
| Total | 100% | w/w | Aggregate value of HLB |

value between 7-10

This concentrate can be incorporated into (1) capsules or administered as freeze dried powder, tablets; or mixed with aqueous carriers to form (2) oral liquids, beverages, beverage additive, food additives, sub-lingual liquids, buccal liquids, intranasal liquids, mist or spray for oral or intranasal use; or mixed with (3) excipients for topical or transdermal application; or mixed with (4) excipients for mucosal applications such as oro-buccal patches, films, tablets, suppositories, etc. Applications of current example are (1) increased water solubility of one or more poorly water-soluble ingredients; (2) increased absorption of one or more poorly water-soluble ingredients; (3) increased absorption of one or more water-soluble ingredients; (4) bypass of first pass metabolism.

### Example 4: Concentrate Formulation C

Analogous preconcentrates for Formulation B can be prepared according to Example 1. Formulation C is a concentrate composition containing a mixture of lipid carriers and surface-active agents, and the active ingredients. The lipid carriers and surface-active agents collectively forming the OS as described earlier are processed through the range of sonic frequencies to form a structural conformity to incorporate the active ingredient that is poorly water-soluble. The active ingredient is heated at temperatures between 0° C. to 100° C. dependent on the maximum heat tolerated by OS as well as active ingredient without decomposing due to heat. Upon heating, the active ingredient is solubilized into the OS to form a clear solution. This concentrate of water-soluble composition containing OS with active can be directly administered as capsules or as freeze-dried powder to be mixed with aqueous liquids within the gastrointestinal tract or be mixed with water based aqueous carrier to form a gel, cream or clear solution. The lipids carrier can be a whole or fractionated form of peanut oil, coconut oil, castor oil, olive oil, hemp seed oil, sesame oil, fish oil, sunflower oil, essential oils, and so on depending on the miscibility of the active ingredients. For example, these include a fractionated form of medium chain glycerides of capric acid, caprylic acid and lauric acid extracted from coconut oil, palm oil, cannabis seed oil and sesame oil in varying ratios as well as a fractionated form of long chain glycerides of myristic acid, oleic acid, stearic acid, palmitic acid and arachidic acid extracted from olive oil, soybean oil, peanut oil, canola oil and macadamia oil in varying ratios. The other component is a surface-active base having a lipophilic head and a hydrophilic tail to reduce the surface tension and balance the HLB values of lipid carrier and active ingredients intended for forming a nano encapsulated system that solubilize in aqueous medium. Table 4 provides a summary of these compositions, including the ranges of quantities of each component.

TABLE 4
Summary of the components and their quantities (% w/w)
of Example Concentrate Formulation C

| Component | % w/w | | |
|---|---|---|---|
| Hemp extract (or | 0.1-90% | w/w | Having an HLB value |
| equivalent active | | | of 0-7 |
| ingredients) | | | |
| Lipophilic component | 0.1-99.9% | w/w | Having an HLB value |
| (fractionated form of olive | | | of less than 0-7 |
| oil, soybean oil, peanut | | | |
| oil, canola oil and | | | |
| macadamia oil, coconut | | | |
| oil, palm oil, cannabis | | | |
| seed oil and sesame oil) | | | |
| Surface active agent | 0.1-99.9% | w/w | Having am HLB value |
| | | | of between 10-13 |
| Total | 100% | w/w | Aggregate value of HLB |

value between 7-10

This concentrate can be incorporated into (1) the capsules or administered as freeze dried powder, tablets; or mixed with aqueous carriers to form (2) oral liquids, beverages, beverage additive, food additives, sub-lingual liquids, buccal liquids, intranasal liquids, mist or spray for oral or intranasal use; or mixed with (3) excipients for topical or transdermal application; or mixed with (4) excipients for mucosal applications such as oro-buccal patches, films, tablets, suppositories, etc. Applications of current example are (1) increased water solubility of one or more poorly water-soluble ingredients; (2) increased absorption of one or more poorly water-soluble ingredients; (3) increased absorption of one or more water-soluble ingredients; (4) bypass first pass metabolism.

### Example 5: Water Solubility and Permeability

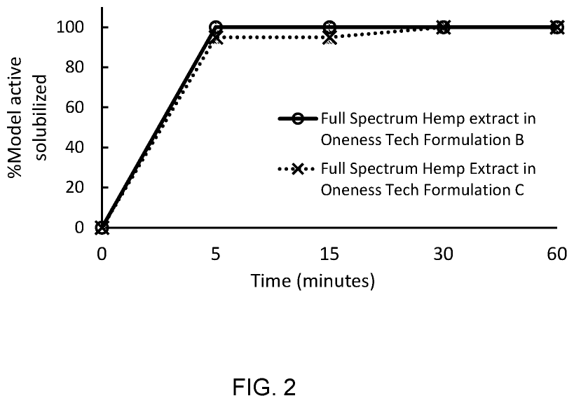

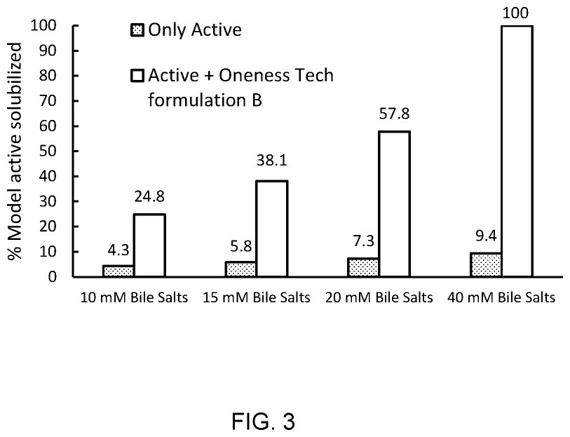

The increased water solubility of a poorly water-soluble model drug (probucol) incorporated into Formulations B and C using the technology of the present invention as a delivery vehicle is shown in FIG. 2 and FIG. 3. Probucol corresponds to the IUPAC name 4,4′-[Propane-2,2-diylbis(thio)]bis(2,6-di-tert-butylphenol), the CAS Registry number 23288-49-5, the chemical formula C₃₁H₄₈O₂S₂, and is sold under the Trade name Lorelco having 5 nanograms per ml solubility in water.

The graph in FIG. 2 shows a 100% solubilization of the model drug in the simulated gastrointestinal aqueous medium when formulated in Formulations B and C.

The graph in FIG. 3 shows an increase in the percentage of drug solubilization in the presence of bile salts when incorporated in the OS preconcentrate Formulation B of the present invention versus without. The main biological function of bile salts is to solubilize dietary lipids and thus greatly accelerate their absorption. See, Hofmann, A. F. (1987). Bile salts as biological surfactants. Colloids and Surfaces (30.1), 45-173. While bile salts help enhance the solubilization of lipophilic actives as well, the application of present invention can further amplify the solubilization capacity of bile salts. The bile salts are the body's natural surface-active agents that help in emulsification of poorly water-soluble molecules thus facilitating absorption through the gut membrane. The present invention has shown a clearly improved solubilization capacity of the active model drug.

The overall permeability characteristics of the small intestine have been widely investigated. Particular emphasis has been given to the influence of physicochemical factors such as acid strength, lipophilicity, and solubility on the intestinal absorption of chemical compounds. It is generally recognized that the epithelial layer, is the most important biological barrier to transmucosal transport of active from gut into the blood stream and concluded that Caco-2 cells grown on collagen-coated polycarbonate mem-branes should represent a valuable transport model system for the small intestinal epithelium. See, Hidalgo, I. J. (1989). Characterization of the human colon carcinoma cell line (Caco-2) as a model system for intestinal epithelial permeability. Gastroenterology (96.2), 736-749. Transport of actives across the intestinal epithelium can occur transcellularly or paracellularly. The extent to which these pathways contribute to the overall flux of the active drug depends upon the environment of the gastrointestinal tract and the physicochemical parameters of the active. The unionized species of the drug actives could partition into the cell membrane, and diffuse across the cell (transcellular transport), whereas, both unionized and ionized species could diffuse across the tight junctions (paracellular transport). Generally, hydrophilic actives are passively transported through the paracellular route, while the more lipophilic solutes use the transcellular route. The Caco-2 cell culture model, is an established model for studying intestinal absorption of actives through paracellular or transcellular pathways. See, Pade, V. a. (1997). Estimation of the relative contribution of the transcellular and paracellular pathway to the transport of passively absorbed drugs in the Caco-2 cell culture model. Pharmaceutical research (14.9), 1210-1215. The measure of paracellular transport is measured by tagging a radiolabeled mannitol along with the active and with or without the present invention. The measure of transcellular transport was measured by tagging a radiolabeled propranolol. See, Cogburn, J. N. (1991). A model of human small intestinal absorptive cells. 1. Transport barrier. Pharmaceutical research (8.2), 210-216.

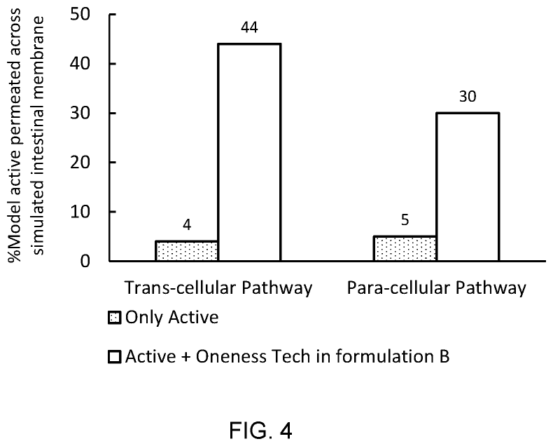

FIG. 4 shows the results for comparing the intestinal permeability using the simulated intestinal model (Caco-2 cell monolayers) for the model active drug, probucol, formulated with and without the OS preconcentrate Formulation B. The drug permeation was enhanced 10-fold by the present invention through both paracellular and transcellular pathways in simulated intestinal membrane.

### Example 6: Exemplary Visibly Clear Aqueous Compositions, Particle Size Determinations, and Stability

Representative formulations of visibly clear aqueous compositions of the present invention were prepared. Table 5 lists examples of visibly clear aqueous compositions having a water-insoluble or poorly water-soluble active ingredient formulated into a concentrate dispersed in an emulsion. Under one description, each composition comprises a water-insoluble active ingredient(s) having an HLB value of zero to about 7, a lipophilic component(s) having an HLB value of zero to about 7, a surface-active agent(s) (surfactant) having an HLB value from about 10 to about 13, and a water or an aqueous carrier.

For each, the concentrate formulation is listed and the measured samples were prepared by first preparing a preconcentrate comprising the lipophilic component(s) and surface active agent(s) by simple mixing and then adding the water-insoluble or poorly water soluble active(s) to form a concentrate. Next, the desired mass of the concentrate is combined with the desired volume (amount) of (deionized) water with simple mixing. The amount of concentrate added to the aqueous carrier (water) is given in the third column of Table 5. The weight percentages of the water insoluble or poorly water soluble active(s) in the aqueous composition are given with respect to the concentrate in the “concentrate formulation” entry (column 4), unless otherwise specified. The particle sizes of the resultant compositions were determined by DLS and are reported in three metrics—particle size, D90, and D50. The reported particle size corresponds to the peak value of the population distribution (i.e. the mode), averaged over samples measured in triplicate. The final column of Table 5 lists the concentration of active in the composition.

TABLE 5
Exemplary Formulations

| | | AMOUNT | CONCEN- |
|---|---|---|---|
| | | OF | TRATION |
| | | CONCEN- | OF ACTIVE |
| | | TRATE IN | IN |
| SAMPLE | CONCENTRATE | AQUEOUS | AQUEOUS |
| ID | FORMULATION | CARRIER | CARRIER |
| O-1 | Concentrate with 10% w/w | 100 mg/ml | 10 mg/ml |
| | CBD isolate active, | | |
| | Surfactant B | | |
| O-2 | Concentrate with 10% w/w | 160 mg/ml | 10 mg/ml |
| | Full spectrum hemp extract | | |
| | active (Corresponding to | | |
| | 7% w/w CBD), Surfactant B | | |
| O-3 | Concentrate with 5% w/w | 100 mg/ml | 5 mg/ml |
| | Melatonin active, | | |
| | Surfactant B | | |
| O-4 | Concentrate with 25% w/w | 40 mg/ml | 10 mg/ml |
| | Fish oil active, Surfactant B | | |
| O-5 | Concentrate with 5% w/w | 100 mg/ml | 5 mg/ml |
| | Turmeric active, Surfactant B | | |
| O-6 | Concentrate with 10% | 100 mg/ml | 10 mg/ml |
| | Astaxanthin active, | | |
| | Surfactant B | | |
| O-7 | Concentrate with 16.66% w/w | 60 mg/ml | 10 mg/ml |
| | Full spectrum hemp extract | | |
| | active (Corresponding to | | |
| | 11.66% w/w CBD), | | |
| | Surfactant A | | |
| O-8 | Concentrate with 16.66% w/w | 88 mg/L | 14 mg/L |
| | Cannabis extract A active | | |
| | (Corresponding to | | |
| | 13.33% w/w THC), | | |
| | Surfactant A | | |
| O-9 | Concentrate with 16.66% w/w | 88 mg/L | 14 mg/L |
| | Cannabis extract B active | | |
| | (Corresponding to | | |
| | 13.33% w/w THC), | | |
| | Surfactant A | | |
| O-10 | OS Blank formulation without | 90 mg/ml | NA |
| | active, Surfactant A | | |
| | (Preconcentrate) | | |

DLS results obtained for freshly prepared samples, 1 month stability, and 3 month stability are presented in Table 6. Representative DLS distributions for Sample 0-2 are shown in FIG. 5. Three replicates of Sample 0-2 were measured and labeled as sample 2-1, 2-2, and 2-3. This representative system is considered to be monodisperse, as the minor features beyond 10³ nm are considered to be instrument artifacts and/or outliers. In both monodisperse and polydisperse systems, the particle size (mode) may be larger than or smaller than the D50 and/or D90 values, which may account for particle sizes on either side of the mode. In polydisperse systems, the distribution may involve additional minor components at larger or smaller hydrodynamic radius compared to the mode, that contribute to the D50 and D90 values. All samples were visibly transparent when initially prepared, at one month, and after three months stability.

TABLE 6
DLS Measurement Results

| | Freshly Prepared | | | 1 Month Stability | | | 3 Month Stability | | |
|---|---|---|---|---|---|---|---|---|---|
| SAMPLE | Particle Size | D90 | D50 | Particle Size | D90 | D50 | Particle Size | D90 | D50 |
| ID | (nm) | (nm) | (nm) | (nm) | (nm) | (nm) | (nm) | (nm) | (nm) |
| O-1 | 26 | 17.1 | 10.1 | 21 | 19 | 9 | 17.2 | 61.5 | 29.8 |
| O-2 | 10.4 | 17.8 | 12.6 | 11 | 36 | 12 | 12.8 | 65.1 | 44 |
| O-3 | 43.3 | 16.2 | 9.1 | 21.2 | 31 | 11 | 33.7 | 150 | 101.6 |
| O-4 | 40.3 | 16.7 | 9.2 | 36.3 | 58 | 21 | 34.2 | 76.9 | 44.1 |
| O-5 | 12 | 16.7 | 9.2 | 12.9 | 68.7 | 55 | 12.4 | 113.8 | 14.5 |
| O-6 | 15.9 | 16.2 | 9.0 | 12.9 | 293 | 95 | 12.6 | 457.5 | 292 |
| O-7 | 8.9 | 16.3 | 9.5 | 12.7 | 101 | 16 | 12.1 | 33.4 | 19.1 |
| O-8 | 8.9 | 16.3 | 9.5 | 120.7 | 175 | 58 | 63 | 77.1 | 65.2 |
| O-9 | 22.8 | 15.6 | 9.4 | 63.1 | 142 | 107 | 51.5 | 113.8 | 86.1 |
| O-10 | 10.1 | 15.8 | 9.8 | 14.5 | 46 | 16 | 14.8 | 49.3 | 29.9 |

The formulation corresponding to sample O-7, was also prepared and measured as two additional, separately prepared samples (samples O-11, and O-12). Samples O-11 and O-12 were measured over an extended period of time to yield both 3 month, and 6 month data, as shown in Table 7. Some time points were not measured for each sample. As can be seen from this comparison, small particles are maintained for at least 6 months, indicating long-term stability of the visibly clear aqueous compositions. All samples were visibly transparent when initially prepared, and at one month, after three months, and after six months stability.

TABLE 7
DLS Measurement Results for samples O-7, O-11, and O-12.

| | | Sample | Sample | Sample |
|---|---|---|---|---|
| Month | Parameter | O-7 | O-11 | O-12 |
| 0 | Particle Size (nm) | 8.9 | — | — |
| | D90 (nm) | 16.3 | — | — |
| | D50 (nm) | 9.5 | — | — |
| 1 | Particle Size (nm) | 12.7 | — | — |
| | D90 (nm) | 101 | — | — |
| | D50 (nm) | 16 | — | — |
| 3 | Particle Size (nm) | 12.1 | 15.3 | 44.9 |
| | D90 (nm) | 33.4 | 26.7 | 61.6 |
| | D50 (nm) | 19.1 | 17.1 | 46.6 |
| 6 | Particle Size (nm) | — | 26.6 | 57.4 |
| | D90 (nm) | — | 77 | 77 |
| | D50 (nm) | — | 35.5 | 58.3 |

### Example 7: Exemplary Concentrate Formulations

Table 8 provides for exemplary concentrate formulations. The formulations may comprise different chain lengths and ratios for each of the lipophilic component(s) and the surface active agent(s), and varying ratios of esterification (mono-, di-, and/or tri-glycerides) are contemplated. Any active or actives falling under the scope of the disclosure may be substituted or added to the exemplary formulations. Components of each of the below formulations, and any disclosed herein, may be interchanged. Further additives, excipients, stabilizers, impurities, moisture, adventitious materials, or other components may be added to or present in the concentrate.

The concentrate formulations are prepared by first producing a mixture of an amount of the lipophilic component(s) and an amount of the surface-active agent(s) by simple mixing. In some embodiments, sonic energy input may be used before, after, with or instead of simple mixing to produce the preconcentrate. An amount of the water insoluble or poorly water-soluble active ingredient(s) is then added to the preconcentrate to produce the concentrate by simple mixing. The concentrates may be further mixed with water or an aqueous carrier to produce a visibly clear aqueous dispersion.

TABLE 8
Concentrate Examples

| Formu- | | | Water Insoluble or |
|---|---|---|---|
| lation | Lipophilic | Surface-Active | Poorly Water Soluble |
| No. | Component(s) | Agent(s) | Active Ingredient(s) |
| 1 | glycerides, C14-18 | Sucrose Stearate | Cannabis Extract |
| | and C16-22- | (25-75%) | (5-40%) |
| | unsaturated mono- | | |
| | and di- | | |
| | (20-70%) | | |
| 2 | Glycerides, C14-18 | PEG-8 Castor Oil | Melatonin |
| | and C16-22- | (10-25%) | (5-40%) |
| | unsaturated mono- | PEG-35 Castor | |
| | and di- | Oil | |
| | (10-40%) | (0-25%) | |
| | | PEG-60 Castor | |
| | | Oil | |
| | | (10-25%) | |
| 3 | glycerides, C14-18 | Sucrose Stearate | Astaxanthin |
| | and C16-22- | (25-75%) | (5-40%) |
| | unsaturated mono- | | |
| | and di- | | |
| | (20-70%) | | |
| 4 | (1-hexadecanoyloxy- | Sucrose Stearate | Astaxanthin |
| | 3-hydroxypropan-2-yl) | (10-40%) | (5-40%) |
| | octadecenoate | PEG-8 Castor Oil | |
| | (20-70%) | (10-40%) | |
| 5 | glycerides, C16-18 | Polysorbate 80 | CBD Isolate |
| | and C18-unsaturated | (25-75%) | (5-40%) |
| | mono-, di and tri- | | |
| | (20-70%) | | |
| 6 | glycerides, C14-18 | PEG-8 Castor Oil | Astaxanthin |
| | and C16-22- | (25-75%) | (5-40%) |
| | unsaturated mono- | | |
| | and di- | | |
| | (20-70%) | | |
| 7 | Glycerides, C14-18 | PEG-8 Castor Oil | Full Spectrum Hemp |
| | and C16-22- | (10-25%) | Extract |
| | unsaturated mono- | PEG-35 Castor | (5-40%) |
| | and di- | Oil | |
| | (10-40%) | (0-25%) | |
| | | PEG-60 Castor | |
| | | Oil | |
| | | (10-25%) | |
| 8 | glycerides, C16-18 | Polysorbate 20 | Melatonin |
| | and C18-unsaturated | (25-75%) | (5-40%) |
| | mono-, di and tri- | | |
| | (20-70%) | | |
| 9 | Glycerides, C14-18 | PEG-8 Castor Oil | Cannabis Extract |
| | and C16-22- | (10-25%) | (5-40%) |
| | unsaturated mono- | PEG-35 Castor | |
| | and di- | Oil | |
| | (10-40%) | (0-25%) | |
| | | PEG-60 Castor | |
| | | Oil | |
| | | (10-25%) | |
| 10 | glycerides C16-18 and | Polysorbate 20 | Full Spectrum Hemp |
| | C18-unsaturated | (25-75%) | Extract |
| | mono- | | (5-40%) |
| | (20-70%) | | |
| 11 | glycerides, C8-18 | Sucrose Stearate | CBD Isolate |
| | (20-70%) | (0-40%) | (5-40%) |
| | | PEG-40 Castor | |
| | | Oil | |
| | | (0-40%) | |
| 12 | Glycerides, C14-18 | PEG-8 Castor Oil | Fish oil |
| | and C16-22- | (10-25%) | (5-40%) |
| | unsaturated mono- | PEG-35 Castor | |
| | and di- | Oil | |
| | (10-40%) | (0-25%) | |
| | | PEG-60 Castor | |
| | | Oil | |
| | | (10-25%) | |
| 13 | glycerides, C14-18 | PEG-8 Castor Oil | Melatonin |
| | and C16-22- | (25-75%) | (5-40%) |
| | unsaturated mono- | | |
| | and di- | | |
| | (20-70%) | | |
| 14 | (1-hexadecanoyloxy- | Sucrose Stearate | Cannabis Extract |
| | 3-hydroxypropan-2-yl) | (10-40%) | (5-40%) |
| | octadecenoate | PEG-8 Castor Oil | Melatonin |
| | (20-70%) | (10-40%) | (5-40%) |
| 15 | glycerides C16-18 and | Polysorbate 20 | Melatonin |
| | C18-unsaturated | (25-75%) | (5-40%) |
| | mono- | | |
| | (20-70%) | | |
| 16 | glycerides, C14-18 | Sucrose Stearate | CBD Isolate |
| | and C16-22- | (25-75%) | (5-40%) |
| | unsaturated mono- | | |
| | and di- | | |
| | (20-70%) | | |
| 17 | Cocoglycerides | Sucrose Stearate | Melatonin |
| | (5-40%) | (10-40%) | (5-40%) |
| | | PEG-8 Castor Oil | |
| | | (0-40%) | |
| 18 | (1-hexadecanoyloxy- | Sucrose Stearate | Fish oil |
| | 3-hydroxypropan-2-yl) | (10-40%) | (5-40%) |
| | octadecenoate | PEG-8 Castor Oil | |
| | (20-70%) | (10-40%) | |
| 19 | Glyceryl monooleate | Sucrose Stearate | Cannabis Extract |
| | (5-40%) | (10-40%) | (5-40%) |
| | Glycerides, C16-18 | PEG-8 Castor Oil | Melatonin |
| | and C18-unsaturated | (10-40%) | (5-40%) |
| | mono-, di and tri-; | | |
| | (5-40%) | | |
| 20 | Glyceryl monooleate | Sucrose Stearate | CBD Isolate |
| | (5-40%) | (10-40%) | (5-40%) |
| | Glycerides, C16-18 | PEG-8 Castor Oil | |
| | and C18-unsaturated | (10-40%) | |
| | mono-, di and tri-; | | |
| | (5-40%) | | |
| 21 | C8/C10 | Sucrose laurate | CBD Isolate |
| | mono/diglycerides | (10-40%) | (5-40%) |
| | (20-70%) | Sodium | |
| | | Taurocholate | |
| | | (10-40%) | |
| 22 | glycerides C16-18 and | Polysorbate 80 | Fish Oil |
| | C18-unsaturated | (25-75%) | (5-40%) |
| | mono- | | |
| | (20-70%) | | |
| 23 | C8/C10 | Macrogol Stearic | Full Spectrum Hemp |
| | mono/diglycerides | Acid | Extract |
| | (0-40%) | (25-75%) | (5-40%) |
| | C18 triglycerides | | |
| | (1,2,3-tri(cis-9- | | |
| | octadecenoyl)glycerol) | | |
| | (0-40%) | | |
| 24 | glycerides C16-18 and | Polysorbate 20 | CBD Isolate |
| | C18-unsaturated | (25-75%) | (5-40%) |
| | mono- | | |
| | (20-70%) | | |
| 25 | Glycerides, C14-18 | PEG-8 Castor Oil | CBD Isolate |
| | and C16-22- | (10-25%) | (5-40%) |
| | unsaturated mono- | PEG-35 Castor | |
| | and di- | Oil | |
| | (10-40%) | (0-25%) | |
| | | PEG-60 Castor | |
| | | Oil | |
| | | (10-25%) | |
| 26 | glycerides, C16-18 | Polysorbate 80 | Melatonin |
| | and C18-unsaturated | (25-75%) | (5-40%) |
| | mono-, di and tri- | | |
| | (20-70%) | | |
| 27 | (1-hexadecanoyloxy- | Sucrose Stearate | Cannabis Extract |
| | 3-hydroxypropan-2-yl) | (10-40%) | (5-40%) |
| | octadecenoate | PEG-8 Castor Oil | CBD Isolate |
| | (20-70%) | (10-40%) | (5-40%) |
| 28 | Glyceryl monooleate | Sucrose Stearate | Cannabis Extract |
| | (20-70%) | (10-40%) | (5-40%) |
| | | PEG-8 Castor Oil | |
| | | (10-40%) | |
| 29 | glycerides, C14-18 | Sucrose Stearate | Melatonin |
| | and C16-22- | (25- 75%) | (5-40%) |
| | unsaturated mono- | | |
| | and di- | | |
| | (20-70%) | | |
| 30 | C18 monoglycerides | PEG-35 Castor | Full Spectrum Hemp |
| | (glyceryl monooleate) | Oil (25-75%) | Extract |
| | (0-40%) | | (5-40%) |
| | C18 diglycerides | | |
| | (glyceryl dioleate) | | |
| | (0-40%) | | |
| | C18 triglycerides | | |
| | (1,2,3-tri(cis-9- | | |
| | octadecenoyl)glycerol) | | |
| | (0-40%) | | |
| 31 | (1-hexadecanoyloxy- | Sucrose Stearate | Full Spectrum Hemp |
| | 3-hydroxypropan-2-yl) | (10-40%) | Extract |
| | octadecenoate | PEG-8 Castor Oil | (5-40%) |
| | (20-70%) | (10-40%) | |
| 32 | C8/C10 | Sucrose laurate | Full Spectrum Hemp |
| | mono/diglycerides | (10-40%) | Extract |
| | (20-70%) | Sucrose Stearate | (5-40%) |
| | | (10-40%) | |
| 33 | C8/C10 | PEG-40 Castor | Full Spectrum Hemp |
| | mono/di/triglycerides | Oil (25-75%) | Extract |
| | (20-70%) | | (5-40%) |
| 34 | Cocoglycerides | Sucrose Stearate | Fish oil |
| | (5-40%) | (0-40%) | (5-40%) |
| | | PEG-8 Castor Oil | |
| | | (0-40%) | |
| 35 | glycerides, C16-18 | Sucrose laurate | Full Spectrum Hemp |
| | and C18-unsaturated | (25-75%) | Extract |
| | mono-, di and tri- | | (5-40%) |
| | (10-40%) | | |
| | C8/C10 | | |
| | mono/diglycerides | | |
| | (10-40%) | | |
| 36 | glycerides, C14-18 | PEG-8 Castor Oil | Fish oil |
| | and C16-22- | (25-75%) | (5-40%) |
| | unsaturated mono- | | |
| | and di- | | |
| | (20-70%) | | |
| 37 | (1-hexadecanoyloxy- | Sucrose Stearate | CBD Isolate |
| | 3-hydroxypropan-2-yl) | (10-40%) | (5-40%) |
| | octadecenoate | PEG-8 Castor Oil | |
| | (20-70%) | (10-40%) | |
| 38 | glycerides, C16-18 | Polysorbate 20 | Clorthiazole |
| | and C18-unsaturated | (25-75%) | (5-40%) |
| | mono-, di and tri- | | |
| | (20-70%) | | |
| 39 | Cocoglycerides | Sucrose Stearate | Cannabis Extract |
| | (5-40%) | (10-40%) | (5-40%) |
| | | PEG-8 Castor Oil | |
| | | (0-40%) | |
| 40 | C8/C10 | Sucrose laurate | CBD Isolate |
| | mono/diglycerides | (10-40%) | (5-40%) |
| | (20-70%) | Sucrose Stearate | |
| | | (10-40%) | |
| 41 | C18 monoglycerides | PEG-40 Castor | Full Spectrum Hemp |
| | (glyceryl monooleate) | Oil | Extract |
| | (0-40%) | (25-75%) | (5-40%) |
| | C18 diglycerides | | |
| | (glyceryl dioleate) | | |
| | (0-40%) | | |
| | C18 triglycerides | | |
| | (1,2,3-tri(cis-9- | | |
| | octadecenoyl)glycerol) | | |
| | (0-40%) | | |
| 42 | Glyceryl monooleate | Sucrose Stearate | Astaxanthin |
| | (20-70%) | (10-40%) | (5-40%) |
| | | PEG-8 Castor Oil | |
| | | (10-40%) | |
| 43 | glycerides, C16-18 | Sucrose | CBD Isolate |
| | and C18-unsaturated | octadecanoate | (5-40%) |
| | mono-, di and tri- | (25-75%) | |
| | (10-40%) | | |
| | C8/C10 | | |
| | mono/diglycerides | | |
| | (10-40%) | | |
| 44 | Glyceryl monooleate | Sucrose Stearate | Cannabis Extract |
| | (5-40%) | (10-40%) | (5-40%) |
| | Glycerides, C16-18 | PEG-8 Castor Oil | CBD Isolate |
| | and C18-unsaturated | (10-40%) | (5-40%) |
| | mono-, di and tri-; | | |
| | (5-40%) | | |
| 45 | glycerides, C16-18 | Sucrose | Full Spectrum Hemp |
| | and C18-unsaturated | octadecanoate | Extract |
| | mono-, di and tri- | (25-75%) | (5-40%) |
| | (10-40%) | | |
| | C8/C10 | | |
| | mono/diglycerides | | |
| | (10-40%) | | |
| 46 | glycerides, C16-18 | Sucrose laurate | Melatonin |
| | and C18-unsaturated | (0-40%) | (5-40%) |
| | mono-, di and tri- | Macrogol Stearic | |
| | (10-40%) | Acid | |
| | C8/C10 | (0-40%) | |
| | mono/diglycerides | | |
| | (10-40%) | | |
| 47 | C8/C10 | Sucrose laurate | Melatonin |
| | mono/diglycerides | (10-40%) | (5-40%) |
| | (20-70%) | Sodium | |
| | | Taurocholate | |
| | | (10-40%) | |
| 48 | Glyceryl monooleate | Sucrose Stearate | Melatonin |
| | (5-40%) | (10-40%) | (5-40%) |
| | Glycerides, C16-18 | PEG-8 Castor Oil | |
| | and C18-unsaturated | (10-40%) | |
| | mono-, di and tri-; | | |
| | (5-40%) | | |
| 49 | glycerides C16-18 and | Polysorbate 80 | Full Spectrum Hemp |
| | C18-unsaturated | (25-75%) | Extract |
| | mono- | | (5-40%) |
| | (20-70%) | | |
| 50 | C8/C10 | PEG-40 Castor | Full Spectrum Hemp |
| | mono/diglycerides | Oil (25-75%) | Extract |
| | (0-40%) | | (5-40%) |
| | C18 triglycerides | | |
| | (1,2,3-tri(cis-9- | | |
| | octadecenoyl)glycerol) | | |
| | (0-40%) | | |
| 51 | Glyceryl monooleate | Sucrose Stearate | Full Spectrum Hemp |
| | (5-40%) | (10-40%) | Extract |
| | Glycerides, C16-18 | PEG-8 Castor Oil | (5-40%) |
| | and C18-unsaturated | (10-40%) | |
| | mono-, di and tri-; | | |
| | (5-40%) | | |
| 52 | Glyceryl monooleate | Sucrose Stearate | CBD Isolate |
| | (20-70%) | (10-40%) | (5-40%) |
| | | PEG-8 Castor Oil | |
| | | (10-40%) | |
| 53 | Glycerides, C14-18 | PEG-8 Castor Oil | Astaxanthin |
| | and C16-22- | (10-25%) | (5-40%) |
| | unsaturated mono- | PEG-35 Castor | |
| | and di- | Oil | |
| | (10-40%) | (0-25%) | |
| | | PEG-60 Castor | |
| | | Oil | |
| | | (10-25%) | |
| 54 | C8/C10 | Sucrose laurate | Full Spectrum Hemp |
| | mono/diglycerides | (10-40%) | Extract |
| | (20-70%) | Sodium | (5-40%) |
| | | Taurocholate | |
| | | (10-40%) | |
| 55 | Cocoglycerides | Sucrose Stearate | Full Spectrum Hemp |
| | (5-40%) | (10-40%) | Extract |
| | | PEG-8 Castor Oil | (5-40%) |
| | | (0-40%) | |
| 56 | C8/C10 | PEG-35 Castor | Full Spectrum Hemp |
| | mono/di/triglycerides | Oil (25-75%) | Extract |
| | (20-70%) | | (5-40%) |
| 57 | glycerides, C14-18 | PEG-8 Castor Oil | Full Spectrum Hemp |
| | and C16-22- | (25-75%) | Extract |
| | unsaturated mono- | | (5-40%) |
| | and di- | | |
| | (20-70%) | | |
| 58 | glycerides, C14-18 | Sucrose Stearate | Full Spectrum Hemp |
| | and C16-22- | (25-75%) | Extract |
| | unsaturated mono- | | (5-40%) |
| | and di- | | |
| | (20-70%) | | |
| 59 | glycerides, C16-18 | Sucrose | Melatonin |
| | and C18-unsaturated | octadecanoate | (5-40%) |
| | mono-, di and tri- | (25-75%) | |
| | (10-40%) | | |
| | C8/C10 | | |
| | mono/diglycerides | | |
| | (10-40%) | | |
| 60 | (1-hexadecanoyloxy- | Sucrose Stearate | Chlorthiazole |
| | 3-hydroxypropan-2-yl) | (10-40%) | (5-40%) |
| | octadecenoate | PEG-8 Castor Oil | |
| | (20-70%) | (10-40%) | |
| 61 | Cocoglycerides | Sucrose Stearate | Astaxanthin |
| | (5-40%) | (10-40%) | (5-40%) |
| | | PEG-8 Castor Oil | |
| | | (0-40%) | |
| 62 | C8/C10 | PEG-35 Castor | Full Spectrum Hemp |
| | mono/diglycerides | Oil (25-75%) | Extract |
| | (0-40%) | | (5-40%) |
| | C18 triglycerides | | |
| | (1,2,3-tri(cis-9- | | |
| | octadecenoyl)glycerol) | | |
| | (0-40%) | | |
| 63 | Glyceryl monooleate | Sucrose Stearate | Fish oil |
| | (5-40%) | (10-40%) | (5-40%) |
| | Glycerides, C16-18 | PEG-8 Castor Oil | |
| | and C18-unsaturated | (10-40%) | |
| | mono-, di and tri-; | | |
| | (5-40%) | | |
| 64 | Glyceryl monooleate | Sucrose Stearate | Cannabis Extract |
| | (5-40%) | (10-40%) | (5-40%) |
| | Glycerides, C16-18 | PEG-8 Castor Oil | Full Spectrum Hemp |
| | and C18-unsaturated | (10-40%) | Extract |
| | mono-, di and tri-; | | (5-40%) |
| | (5-40%) | | |
| 65 | Glyceryl monooleate | Sucrose Stearate | Astaxanthin |
| | (5-40%) | (10-40%) | (5-40%) |
| | Glycerides, C16-18 | PEG-8 Castor Oil | |
| | and C18-unsaturated | (10-40%) | |
| | mono-, di and tri-; | | |
| | (5-40%) | | |
| 66 | Glyceryl monooleate | Sucrose Stearate | Cannabis Extract |
| | (5-40%) | (10-40%) | (5-40%) |
| | Glycerides, C16-18 | PEG-8 Castor Oil | |
| | and C18-unsaturated | (10-40%) | |
| | mono-, di and tri-; | | |
| | (5-40%) | | |
| 67 | C18 monoglycerides | Macrogol Stearic | Full Spectrum Hemp |
| | (glyceryl monooleate) | Acid | Extract |
| | (0-40%) | (25-75%) | (5-40%) |
| | C18 diglycerides | | |
| | (glyceryl dioleate) | | |
| | (0-40%) | | |
| | C18 triglycerides | | |
| | (1,2,3-tri(cis-9- | | |
| | octadecenoyl)glycerol) | | |
| | (0-40%) | | |
| 68 | glycerides, C16-18 | Sucrose laurate | CBD Isolate |
| | and C18-unsaturated | (25-75%) | (5-40%) |
| | mono-, di and tri- | | |
| | (10-40%) | | |
| | C8/C10 | | |
| | mono/diglycerides | | |
| | (10-40%) | | |
| 69 | C14-18 and C16-18- | Glucose | Clorthiazole |
| | unsaturated mono-, | octadecenoate | (5-40%) |
| | di-and tri-; glycerides | (25-75%) | |
| | (20-70%) | | |
| 70 | (1-hexadecanoyloxy- | Sucrose Stearate | Melatonin |
| | 3-hydroxypropan-2-yl) | (10-40%) | (5-40%) |
| | octadecenoate | PEG-8 Castor Oil | |
| | (20-70%) | (10-40%) | |
| 71 | Cocoglycerides | Sucrose Stearate | CBD Isolate |
| | (5-40%) | (10-40%) | (5-40%) |
| | | PEG-8 Castor Oil | |
| | | (0-40%) | |
| 72 | glycerides, C16-18 | Polysorbate 80 | Full Spectrum Hemp |
| | and C18-unsaturated | (25-75%) | Extract |
| | mono-, di and tri- | | (5-40%) |
| | (20-70%) | | |
| 73 | glycerides, C16-18 | Polysorbate 20 | Astaxanthin |
| | and C18-unsaturated | (25-75%) | (5-40%) |
| | mono-, di and tri- | | |
| | (20-70%) | | |
| 74 | glycerides, C14-18 | PEG-8 Castor Oil | Cannabis Extract |
| | and C16-22- | (25-75%) | (5-40%) |
| | unsaturated mono- | | |
| | and di- | | |
| | (20-70%) | | |
| 75 | Glyceryl monooleate | Sucrose Stearate | Melatonin |
| | (20-70%) | (10-40%) | (5-40%) |
| | | PEG-8 Castor Oil | |
| | | (10-40%) | |
| 76 | Glyceryl monooleate | Sucrose Stearate | Full Spectrum Hemp |
| | (20-70%) | (10-40%) | Extract |
| | | PEG-8 Castor Oil | (5-40%) |
| | | (10-40%) | |
| 77 | Glyceryl monooleate | Sucrose Stearate | Fish oil |
| | (10-40%) | (10-40%) | (5-40%) |
| | | PEG-8 Castor Oil | |
| | | (10-40%) | |
| 78 | glycerides, C8-18 | Sucrose Stearate | Melatonin |
| | (20-70%) | (25-75%) | (5-40%) |
| 79 | glycerides, C8-18 | Sucrose Stearate | Full Spectrum Hemp |
| | (20-70%) | (0-40%) | Extract |
| | | Macrogol Stearic | (5-40%) |
| | | Acid | |
| | | (0-40%) | |
| 80 | glycerides C16-18 and | Polysorbate 80 | CBD Isolate |
| | C18-unsaturated | (25-75%) | (5-40%) |
| | mono- | | |
| | (20-70%) | | |
| 81 | (1-hexadecanoyloxy- | Sucrose Stearate | Full Spectrum Hemp |
| | 3-hydroxypropan-2-yl) | (10-40%) | Extract |
| | octadecenoate | PEG-8 Castor Oil | (5-40%) |
| | (20-70%) | (10-40%) | CBD Isolate |
| | | | (5-40%) |
| 82 | glycerides, C14-18 | Sucrose Stearate | Fish oil |
| | and C16-22- | (25-75%) | (5-40%) |
| | unsaturated mono- | | |
| | and di- | | |
| | (20-70%) | | |
| 83 | (1-hexadecanoyloxy- | Sucrose Stearate | Cannabis Extract |
| | 3-hydroxypropan-2-yl) | (10-40%) | (5-40%) |
| | octadecenoate | PEG-8 Castor Oil | Full Spectrum Hemp |
| | (20-70%) | (10-40%) | Extract |
| | | | (5-40%) |
| 84 | C8/C10 | Macrogol Stearic | Full Spectrum Hemp |
| | mono/di/triglycerides | Acid | Extract |
| | (20-70%) | (25-75%) | (5-40%) |
| 85 | C8/C10 | Sucrose laurate | Melatonin |
| | mono/diglycerides | (10-40%) | (5-40%) |
| | (20-70%) | Sucrose Stearate | |
| | | (10-40%) | |
| 86 | glycerides, C14-18 | PEG-8 Castor Oil | CBD Isolate |
| | and C16-22- | (25-75%) | (5-40%) |
| | unsaturated mono- | | |
| | and di- | | |
| | (20-70%) | | |
| 87 | (1-hexadecanoyloxy- | Sucrose Stearate | Cannabis Extract |
| | 3-hydroxypropan-2-yl) | (10-40%) | (5-40%) |
| | octadecenoate | PEG-8 Castor Oil | |
| | (20-70%) | (10-40%) | |

It is to be understood that the above description is intended to be illustrative, and not restrictive. For example, the above-discussed embodiments can be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the compositions and methods of the present invention, where the term comprises is used with respect to the recited steps of the methods or components of the compositions, it is also contemplated that the compositions and methods consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

What is claimed is:

1. A preconcentrate composition for solubilizing, dispersing, or emulsifying a water-insoluble or poorly water-soluble active ingredient in an aqueous carrier, said preconcentrate composition, comprising:
a. a lipophilic component having an hydrophilic-lipophilic balance (HLB) value of zero to about 7, and
b. a surface-active agent having an HLB value from about 10 to about 13;
(i) wherein the lipophilic component comprises from about 0.1% to about 99.9%, or from about 1% to about 80%, or from about 5% to about 50%, or from about 10% to about 40%, or from about 10% to about 25% by weight of the pre-concentrate;
(ii) wherein the surface active agent comprises from about 0.1% to about 99.9%, or from about 1% to about 80%, or from about 5% to about 50%, or from about 10% to about 40%, or from about 10% to about 25% by weight of the pre-concentrate;
(iii) wherein when said preconcentrate is combined with said water-insoluble or poorly water-soluble active ingredient forms a concentrate having an HLB value from about 7 to about 10;
(iv) wherein said resultant concentrate when combined with an aqueous carrier provides a dispersion in said aqueous carrier, wherein the dispersion comprises particles formed from the concentrate; and
(vi) wherein said resultant dispersion is visibly clear.

2. The preconcentrate according to claim 1 wherein (i) the lipophilic component is selected from the group consisting of plant-based oils, glycerides, waxes, alcohols, hydroalcoholic mixtures, whole and fractionated oil forms of any of the foregoing, and mixtures thereof; or (ii) the lipophilic component is a plant-based oil, or a whole or fractionated oil form thereof, and mixtures thereof; or (iii) the lipophilic component is a plant-based oil is selected from the group consisting of almond oil, avocado oil, borage oil, brazil nut oil, cannabis oil, cannabis-seed oil, canola oil, cashew oil, castor oil, chia seed oil, cocoa butter oil, coconut oil, corn oil, cottonseed oil, flaxseed oil, grape seed oil, hemp seed oil, linseed oil, mustard oil, olive oil, palm oil, peanut oil, pecan oil, peppermint oil, perilla oil, poppy seed oil, rapeseed oil, rice bran oil, safflower oil, sesame oil, sesame seed oil, soybean oil, sunflower oil, vigna munga oil, walnut oil, whole and fractionated oil forms of any of the foregoing, and mixtures thereof.

3. The preconcentrate according to claim 1 wherein the lipophilic component is a glyceride selected from monoglycerides, diglycerides, triglycerides, and mixtures thereof.

4. The preconcentrate according to claim 3 wherein the glyceride is selected from monoglycerides, diglycerides, and triglycerides of C6 to C30 carboxylic acids, and mixtures thereof, wherein the C6 to C30 carboxylic acids are selected from fully saturated carboxylic acids, carboxylic acids having 1, 2, or 3 unsaturated carbon-carbon bonds which can be variously positioned along the carbon skeleton of the carboxylic acid and which can each individually have a cis or trans isomeric configuration, and wherein the C6 to C30 carboxylic acids can be optionally substituted with one or more hydroxyl groups, amino groups, or carbonyl groups, and combinations of these groups.

5. The preconcentrate according to claim 3 wherein the glyceride is selected from the group consisting of those corresponding to the following CAS registry numbers CAS 92045-31-3, CAS 91744-32-0, CAS 85536-07-8, CAS 91052-28-7, CAS 91744-09-1, CAS 91744-13-7, CAS 85251-77-0, CAS 84244-35-9, CAS 85536-06-7, CAS 91744-20-6, CAS 122-32-7, CAS 25496-72-4, and mixtures thereof.

6. The preconcentrate according to claim 3 wherein the glyceride is selected from the group consisting of cocoglycerides; glyceryl caprate (C8-10 mono, di, and triglycerides); glycerides, C14-18 and C16-18-unsaturated mono-, di- and tri-; glycerides C16-18 and C18-unsaturated mono-; glycerides, C14-18 and C16-22-unsaturated mono- and di-; glycerides C16-18 mono- and di-; (1-hexadecanoyloxy-3-hydroxypropan-2-yl) octadecenoate; glycerides, C8-18; glycerides, C16-18 and C18-unsaturated mono-, di and tri-; 1,2,3-tri(cis-9-octadecenoyl)glycerol; glyceryl monooleate; and mixtures thereof.

7. The preconcentrate according to claim 1 wherein the surface-active agent comprises a hydrophilic head group and one or more side chains selected from C10-C30 fatty acids, wherein the surface-active agent is ethoxylated, propoxylated, or mixed ethoxylated/propoxylated.

8. The preconcentrate according to claim 7 wherein the one or more C10-C30 fatty acids each have at least one hydroxy substituent.

9. The preconcentrate according to claim 8 wherein at least one of the hydroxy substituents of the one or more C10-C30 fatty acids is ethoxylated, propoxylated, or mixed ethoxylated/propoxylated.

10. The preconcentrate according to claim 7 wherein at least one or more of the one or more C10-C30 fatty acids each have at least one carbon-carbon unsaturated bond.

11. The preconcentrate according to claim 10 wherein the one or more of the one or more C10-C30 fatty acids have at least one carbon-carbon unsaturated bond in the cis configuration.

12. The preconcentrate according to claim 8 wherein the hydrophilic head group is selected from the group consisting of aliphatic alcohols, aliphatic polyhydric alcohols, saccharides, disaccharides, aliphatic amines, aliphatic polyamines, aliphatic amino alcohols, aliphatic amino polyhydric alcohols, aliphatic polyamino alcohols, aliphatic polyamino polyhydric alcohols, and combinations thereof.

13. The preconcentrate according to claim 12 wherein the hydrophilic head group is selected from the group consisting of ethylene glycol, propylene glycol, 1,3-propane diol, 1,2-butane diol, 1,3-butane diol, 1,4-butane diol, glycerol, glyceraldehyde, 1-hydroxy-2-amino ethane, and combinations thereof.

14. The preconcentrate according to claim 13 wherein the hydrophilic head group is glycerol, and wherein the surface-active agent comprises a mono-, di-, or triglyceride of a C10-C30 fatty acid wherein each C10-C30 fatty acid is independently modified with one or more ethoxy groups, one or more propoxy groups, or a mixture of one or more ethoxy and propoxy groups.

15. The preconcentrate according to claim 13 wherein (i) the hydrophilic head group is covalently bonded to the one or more C10-C30 fatty acids directly via an ester linkage to the C10-C30 fatty acid, and wherein at least one of the hydroxy substituents of at least one of the C10-C30 fatty acid chains is independently modified with one or more ethoxy groups, propoxy groups, or a mixture of one or more ethoxy and propoxy groups; or
(ii) the hydrophilic head group is covalently bonded to the one or more C10-C30 fatty acids indirectly via an ester or ether, linkage to an intervening ethoxy, propopxy, or mixed ethoxy/propoxy group;
or
(ii) the hydrophilic head group is covalently bonded to the one or more C10-C30 fatty acids indirectly via an ester or ether linkage to an intervening ethoxy, propoxy, or mixed ethoxy/propoxy group, and wherein at least one of the hydroxy substituents of at least one of the C10-C30 fatty acid chains is modified with one or more ethoxy groups, propoxy groups, or mixed ethoxy/propoxy groups.

16. The preconcentrate according to claim 7 wherein the surface active agent is selected from the group consisting of CAS 61788-85-0, CAS 188734-82-9, CAS 57176-33-7, CAS 70142-34-6, CAS 68953-20-8, CAS 31835-02-6, CAS 13039-40-2, CAS 61791-12-6, CAS 854374-08-6, CAS 122636-35-5, CAS 122636-36-6, CAS 9005-64-5, CAS 9005-65-6, CAS 145-42-6, CAS 388610-12-6, and mixtures thereof.

17. The preconcentrate according to claim 7 wherein the surface-active agent is selected from the group consisting of sorbitan esters, ethoxylated sorbitan esters, polyalcohols, ethoxylated alky phenols, amine derivatives, amide derivatives, alkylpolyglucosides, ethyleneoxide-propylene-oxide copolymers, thiols or derivatives thereof, poloxamers, pegylated (ethoxylated) fatty acid esters, propoxylated fatty acid esters, mixed ethoxylated/propoxylated fatty acid esters, pegylated (ethoxylated) fatty acid triglycerides, propoxylated fatty acid triglycerides, mixed ethoxylated/propoxylated fatty acid triglycerides, pegylated (ethoxylated) hydroxy substituted fatty acid triglycerides, propoxylated hydroxy substituted fatty acid triglycerides, mixed ethoxylated/propoxylated hydroxy substituted fatty acid triglycerides, wherein said fatty acids are optionally unsaturated, polysorbates, sugar ester, lecithin, bile salts, albumin, alcohols, and mixtures thereof.

18. The preconcentrate according to claim 7 wherein the surface-active agent is selected from the group consisting of ethoxylated castor oil (polyoxyethylene castor oil); RO 40; BY 140; PEG Castor oil; PEG-10 Castor oil, PEG-100 Castor oil, PEG-1 Castor oil, PEG-15 Castor oil, PEG-2 Castor oil, PEG-20 Castor oil, PEG-200 Castor oil, PEG-25 Castor oil, PEG-26 Castor oil, PEG-3 Castor oil, PEG-30 Castor oil, PEG-33 Castor oil, PEG-35 Castor oil, PEG-36 Castor oil, PEG-4 Castor oil, PEG-40 Castor oil, PEG-5 Castor oil, PEG-50 Castor oil, PEG-54 Castor oil, PEG-55 Castor oil, PEG-60 Castor oil, PEG-8 Castor oil, PEG-9 Castor oil, polyethoxylated castor oil, polyethylene glycol (100) castor oil, polyethylene glycol (11) castor oil, polyethylene glycol (15) castor oil, polyethylene glycol (25) castor oil, polyethylene glycol (26) castor oil, polyethylene glycol (3) castor oil, polyethylene glycol (30) castor oil, polyethylene glycol (33) castor oil, polyethylene glycol (35) castor oil, polyethylene glycol (5) castor oil, polyethylene glycol (50) castor oil, polyethylene glycol (54) castor oil, polyethylene glycol (55) castor oil, polyethylene glycol (60) castor oil, polyethylene glycol 1000 castor oil, polyethylene glycol 1800 castor oil, polyethylene glycol 200 castor oil, polyethylene glycol 2000 castor oil, polyethylene glycol 400 castor oil, polyethylene glycol 450 castor oil, polyethylene glycol 500 castor oil, polyoxyethylene (10) castor oil, polyoxyethylene (100) castor oil, polyoxyethylene (11) castor oil, polyoxyethylene (15) castor oil, polyoxyethylene (2) castor oil, polyoxyethylene (20) castor oil, polyoxyethylene (200) castor oil, polyoxyethylene (25) castor oil, polyoxyethylene (26) castor oil, polyoxyethylene (3) castor oil, polyoxyethylene (30) castor oil, polyoxyethylene (33) castor oil, polyoxyethylene (35) castor oil, polyoxyethylene (36) castor oil, polyoxyethylene (4) castor oil, polyoxyethylene (40) castor oil, Polyoxyethylene (5) castor oil, polyoxyethylene (50) castor oil, polyoxyethylene (54) castor oil, polyoxyethylene (55) castor oil, polyoxyethylene (60) castor oil, polyoxyethylene (8) castor oil, polyoxyethylene (9) castor oil, and mixtures thereof.

19. A concentrate composition for solubilizing, dispersing, or emulsifying a water-insoluble or poorly water-soluble active ingredient in an aqueous carrier, said concentrate composition, comprising:
a. a water-insoluble active or poorly water-soluble active ingredient having an HLB value of zero to about 7,
b. a lipophilic component having an HLB value of zero to about 7, and
c. a surface-active agent having an HLB value from about 10 to about 13;
(i) wherein the water-insoluble or poorly water-soluble active ingredient comprises from about 0.01% to about 80%, or from about 0.1% to about 50%, or from about 0.5% to about 25%, or from about 1% to about 20% by weight of the concentrate;
(ii) wherein the lipophilic component comprises from about 0.1% to about 99.9%, or from about 1% to about 80%, or from about 5% to about 50%, or from about 10% to about 40%, or from about 10% to about 25% by weight of the concentrate;
(iii) wherein the surface active agent comprises from about 0.1% to about 99.9%, or from about 1% to about 80%, or from about 5% to about 50%, or from about 10% to about 40%, or from about 10% to about 25% by weight of the concentrate;
(iv) wherein said concentrate has an HLB value from about 7 to about 10;
(v) wherein said concentrate when combined with an aqueous carrier provides a dispersion in said aqueous carrier, wherein the dispersion comprises particles formed from the concentrate; and
(v) wherein said resultant dispersion is visibly clear.

20. The concentrate according to claim 19 wherein the water-insoluble or poorly water-soluble active ingredient is selected from the group consisting of essential oils (i.e. also known as plant extracts or botanical extracts), pharmaceutical drug actives, entheogenic plants, mushrooms, psychedelic agents, polypeptides and protein, vitamins, fish oil, milk derivatives, fragrances, flavorings, colorings, sweeteners, taste-enhancers, anti-oxidants, and mixtures thereof.

21. The concentrate according to claim 19 wherein the water-insoluble or poorly water-soluble active ingredient is selected from the group consisting of cannabis extract, hemp oil, human breast milk, cannabinoids, natural phytocannabinoids, organic cannabinoids, endocannabinoids, cannabinoid analogs, cannabinoid derivatives, synthetic cannabinoids, cannabinoid receptor agonists, and mixtures thereof.

22. The concentrate according to claim 21 wherein the cannabinoids are selected from the group consisting of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta-9-tetrahydrocannabivarinic acid (THCVA), delta-9-tetrahydrocannabivarin (THCV), delta-9-tetrahydrocannabiorcolic acid (THCA-C1), delta-9-tetrahydrocannabiorcol (THC-C1), delta-7-cis-iso-tetrahydrocannabivarin, delta-8-tetrahydrocannabinolic acid (.DELTA8-THCA), delta-8-tetrahydrocannabinol (DELTAS-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-metha-no-2H-1-benzoxocin-5-methanol (OH-iso-HHCV), cannabiripsol (CBR) and trihydroxy-delta-9-tetrahydrocannabinol (triOH-THC), and mixtures thereof.

23. A visibly clear aqueous composition of a water-insoluble or poorly water-soluble active ingredient, comprising:
a. a water-insoluble or poorly water soluble active ingredient having an HLB value of zero to about 7,
b. a lipophilic component having an HLB value of zero to about 7,
c. a surface-active agent having an HLB value from about 10 to about 13, and
d. water or an aqueous carrier;
(i) wherein the water-insoluble or poorly water soluble active ingredient comprises from about 0.0001% to about 80%, or from about 0.001% to about 50%, or from about 0.001% to about 25%, or from about 0.01% to about 25%, or from about 0.1% to about 25%, or from about 0.1% to about 10% by weight of the visibly clear aqueous composition;
(ii) wherein the lipophilic component comprises from about 0.001% to about 25%, or from about 0.01% to about 10%, or from about 0.05% to about 5%, or from about 0.1% to about 2%, or from about 0.1% to about 1% by weight of the visibly clear aqueous composition;
(iii) wherein the surface active agent comprises from about 0.001% to about 25%, or from about 0.01% to about 10%, or from about 0.05% to about 5%, or from about 0.1% to about 2%, or from about 0.1% to about 1% by weight of the visibly clear aqueous composition;
(iv) wherein the combination of said water-insoluble active ingredient, said lipophilic component, and said surface-active agent has an HLB value from about 7 to about 10; and
(v) wherein the combination of the water-insoluble or poorly water soluble active ingredient, lipophilic component, and surface-active agent comprise from about 0.002% to about 25%, or from about 0.01% to about 10%, or from about 0.05% to about 5%, or from about 0.1% to about 2%, or from about 0.1% to about 1% by weight of the visibly clear aqueous composition; or
wherein the combination of the water-insoluble or poorly water soluble active ingredient, lipophilic component, and surface-active agent has a concentration from about 20 mg/L to about 250 g/L, or from about 50 mg/L to about 100 g/L, or from about 0.5 g/L to about 10 g/L, or from about 0.75 g/L to about 1.25 g/L in the visibly clear aqueous composition; and
(vi) wherein said visibly clear aqueous composition is a dispersion.

24. The visibly clear aqueous composition according to claim 43 wherein the water or aqueous carrier comprises the remainder of the composition by weight.

25. The visibly clear aqueous composition according to claim 23, wherein the dispersion comprises particles having a distribution of particle sizes, said distribution having a mode less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

26. The visibly clear aqueous composition according to claim 23 wherein the dispersion comprises particles having a distribution of particle sizes, said distribution having a mode in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

27. The visibly clear aqueous composition according to claim 23, wherein the dispersion comprises particles having a distribution of particle sizes, said distribution having a D50 value less than about 250 nm, or less than about 200 nm, or less than about 150 nm, or less than about 100 nm, or less than about 80 nm, or less than about 75 nm, or less than about 50 nm, or less than about 40 nm, or less than about 30 nm, or less than about 25 nm, or less than about 20 nm, or less than about 15 nm, or less than about 12 nm, or less than about 10 nm.

28. The visibly clear aqueous composition according to claim 23, wherein the dispersion comprises particles having a distribution of particle sizes, said distribution having a D50 value in the range from about 8 nm to about 250 nm, or from about 8 nm to about 150 nm, or from about 8 nm to about 100 nm, or from about 8 nm to about 75 nm, or from about 8 nm to about 50 nm, or from about 8 nm to about 40 nm, or from about 8 nm to about 30 nm, or from about 8 nm to about 25 nm, or from about 8 nm to about 20 nm, or from about 8 nm to about 15 nm, or from about 8 nm to about 12 nm.

29. The visibly clear aqueous composition according to claim 23 further comprising a water-soluble compound selected from the group consisting of water-soluble plant extracts, pharmaceutical drug actives, vitamins, fragrances, flavorings, colorings, sweeteners, taste-enhancers, anti-oxidants, and mixtures thereof.

30. The visibly clear aqueous composition according to claim 29 wherein the water-soluble compound is selected from the group consisting of aloe vera extract, green tea extract, stevia leaf extract, and mixtures thereof.
